(19) United States
(12) Patent Application Publication
(10) Pub. No.: US 2022/0226329 A1
(43) Pub. Date: Jul. 21, 2022

(54) METHOD TO INDUCE HEMATOPOIETIC CHIMERISM

(71) Applicant: The General Hospital Corporation, Boston, MA (US)

(72) Inventor: Tatsuo Kawai, Weston, MA (US)

(21) Appl. No.: 17/609,181

(22) PCT Filed: May 8, 2020
(86) PCT No.: PCT/US20/32144

§ 371 Date: Nov. 5, 2021

Related U.S. Application Data
(60) Provisional application No. 62/845,680, filed on May 9, 2019.

(51) Int. Cl. A61K 31/519; A61K 31/635; A61P 37/06; A61K 38/13; A61K 35/28

(52) U.S. Cl. CPC A61K 31/519; A61K 31/635; A61K 2039/505; A61K 38/13; A61K 35/28; A61P 37/06

(57) ABSTRACT

Provided herein are methods for inducing chimerism in a patient. Specifically, provided herein are improved methods for inducing a state in the recipient of allogeneic hematopoietic stem cells such that the lymphohematopoietic system of the recipient comprises a mixture of host and donor cells. Compositions, kits, and systems for use with these methods are also disclosed.

This application claims benefit of priority of U.S. Provisional Application No. 62/845,680, filed on May 9, 2019, the entire contents of which are incorporated herein by reference.

### 1. STATEMENT REGARDING FEDERAL FUNDING

This invention was made with government support under Grant Nos. AI139679 and AI126289 awarded by the National Institutes of Health. The government has certain right in the invention.

### 2. INTRODUCTION

Provided herein are methods for inducing chimerism in a patient. Specifically, provided herein are improved methods for inducing a state in the recipient of allogeneic hematopoietic stem cells such that the lymphohematopoietic system of the recipient comprises a mixture of host and donor cells or only donor cells (hematopoietic). Compositions, kits, and systems for use with these methods are also disclosed.

### 3. BACKGROUND

Complications caused by chronic immunosuppression and chronic rejection of donor graft remains an issue in long-term results of organ transplantation. Among the different existing methods to induce tolerance, very few remain successful due to health issues associated with the conditioning to achieve tolerance. Thus, a need exists for improved methods of inducing tolerance in a recipient's immune system towards the transplant.

Combined kidney and bone marrow transplantation has been described. See, e.g., Kawai et al. 2014, American Journal of Transplantation 14:1599-1611. This study showed that long-term stable kidney allograft survival without maintenance immunosuppression. However, revisions to the conditioning regimen were still needed to improve the clinical outcome. The methods disclosed herein provide an improved treatment regimen to provide consistent tolerance to organ transplantation.

Cippa et al. reported that hematopoietic chimerism and skin allograft tolerance can be achieved without myelosuppressive treatments in mice by using a pro-apoptotic B cell lymphoma-2 (Bcl-2) inhibitor in combination with costimulatory blockade and cyclosporine (CyA) Cippa, P. E., et al. Blood 122, 1669-1677 (2013). Cippa, P. E., et al. Am J Transplant 14, 333-342 (2014). The small molecule, ABT-737 used in the mouse studies by Cippa inhibits Bcl-2, Bcl-xL and Bcl-w but not Mcl-1 and therefore has a selective pro-apoptotic effect on peripheral lymphocytes and platelets, but not on hematopoietic progenitors (Carrington, E. M., et al. BH3 mimetics antagonizing restricted prosurvival Bcl-2 proteins represent another class of selective immune modulatory drugs. Proc Natl Acad Sci USA 107, 10967-10971 (2010).

### 4. SUMMARY

In one aspect, provided herein is a method for inducing hematopoietic chimerism (e.g. mixed chimerism, full donor chimerism) in a patient in need thereof, wherein the method comprises: a) administering an inhibitor of an anti-apoptotic Bcl-2 family member to the patient; b) administering total body irradiation (“TBI”) to the patient; and c) transplanting bone marrow or hematopoietic stem cells (HSCs) from a donor to the patient; such that hematopoietic chimerism is induced in the patient.

In one aspect, provided herein is a method for inducing hematopoietic chimerism or full donor chimerism in a patient in need thereof, wherein the method comprises: a) administering a specific inhibitor of Bcl-2; and b) transplanting bone marrow or HSCs from a donor to the patient; such that hematopoietic chimerism is induced in the patient.

In one aspect, provided herein is a method for inducing hematopoietic chimerism in a patient in need thereof, wherein the method comprises: a) administering a first course of a first inhibitor of an anti-apoptotic Bcl-2 family member to the patient; transplanting bone marrow from a donor to the patient; and b) administering a second course of a second inhibitor of an anti-apoptotic Bcl-2 family member to the patient starting 0, 1, 2, 3, 4, 5, 6, 7, 8, 9, or 10 days after completion of the first course, such that hematopoietic chimerism is induced in the patient. In some embodiments, administering the second course of the second inhibitor of an anti-apoptotic Bcl-2 family member to the patient starts between 2 days to 5 days after completion of the first course. In a certain embodiment, the initial administration of the first inhibitor of an anti-apoptotic Bcl-2 family member to the patient occurs at the same time as the initial administration of the second anti-apoptotic BCL-2 family member to the patient.

In one aspect, provided herein is a method for inducing hematopoietic chimerism in a patient in need thereof, wherein the method comprises administering an inhibitor of an anti-apoptotic Bcl-2 family member to the patient, wherein the patient is not being treated with an anti-CD154 antibody.

In one aspect, provided herein is a method for inducing hematopoietic chimerism in a patient in need thereof, wherein the method comprises administering an inhibitor of an anti-apoptotic Bcl-2 family member to the patient, wherein the inhibitor is not ABT-737. In another aspect, provided herein is a method for inducing hematopoietic chimerism in a patient in need thereof, wherein the method comprises administering an inhibitor of an anti-apoptotic Bcl-2 family member to the patient, wherein the inhibitor is not ABT-263.

In one aspect, provided herein is a method for inducing hematopoietic chimerism in a patient in need thereof, wherein the method comprises: a) administering an inhibitor of an anti-apoptotic Bcl-2 family member to the patient; b) administering total body irradiation (“TBI”) to the patient; and c) transplanting bone marrow cells or peripheral blood stem cells (PBSC) (obtained by leukapheresis) and an organ selected from heart, intestine, kidney, or liver from a donor to the patient.

In one aspect, provided herein is a method for inducing hematopoietic chimerism in a patient in need thereof, wherein the method comprises: a) administering a first course of a first inhibitor of an anti-apoptotic Bcl-2 family member to the patient; b) administering a second course of a second inhibitor of an anti-apoptotic Bcl-2 family member to the patient; and c) transplanting bone marrow from a donor to the patient. In some embodiments, the first and the second inhibitor of an anti-apoptotic Bcl-2 family member are the same. In some embodiments, the first and second inhibitor target the same member of the BCL-2 family of proteins. In some embodiments, the first and second inhibitor target different members of the BCL-2 family of proteins. In some embodiments, the first and the second inhibitor are not the same.

In certain embodiments, hematopoietic chimerism includes, but is not limited to, mixed chimerism and full donor chimerism. In some embodiments, the treatment regimen reduces the risk of Graft versus Host Disease (GVHD) in the recipient. In some embodiments, the treatment regimen reduces the incidence of GVHD by about 5%, 10%, 15%, 20%, 25%, 30%, 35%, 40%, 45%, 50%, 55%, 60%, 65%, 70%, 75%, or 80%, 85%, 95% as compared to no treatment regimen in the recipient. In some embodiments, the treatment regimen reduces the severity and/or degree of GVHD in the recipient by about 5%, 10%, 15%, 20%, 25%, 30%, 35%, 40%, 45%, 50%, 55%, 60%, 65%, 70%, 75%, or 80%, 85%, 95% as compared to no treatment regimen in the recipient. In some embodiments, the dose of TBI is 0.5 Gy, 0.75 Gy, 1.0 Gy, 1.25 Gy, 1.50 Gy, 1.75 Gy, 2.0 Gy, 2.25 Gy, 2.50 Gy, or 3 Gy. In some embodiments, the dose of TBI is 1.5 Gy

In some embodiments, the anti-apoptotic Bcl-2 family member is Bcl-2. In some embodiments, the specific inhibitor of Bcl-2 is venetoclax, oblimersen, PNT2258, or SPC2996. In some embodiments, the specific inhibitor of Bcl-2 is venetoclax. In some embodiments, the inhibitor of an anti-apoptotic BCL-2 family member is a combination of inhibitors of Mcl-1 and Bcl-2. In some embodiments, the inhibitor of Mcl-1 is obatoclax, A-1210477, AMG176, S64315 (MIK665), S63845, or AZD5991. In some embodiments, the inhibitor of Mcl-1 is S63845 or S64315.

In some embodiments, chimerism can be induced without need of myelosuppressive treatments (e.g., cyclophosphamide or TBI).

In some embodiments, the method does not result in neutropenia. In some embodiments, the method results in a nadir (lowest levels) of neutrophil counts no less than 100/mm³, 200/mm³, 300/mm³, 400/mm³, 500/mm³, 600/mm³, 700/mm³, 800/mm³, 900/mm³, 1000/mm³, 1500/mm³, 1600/mm³, 1700/mm³, 1800/mm³, 1900/mm³, 2000/mm³, before the method.

In some embodiments, the method results in a nadir (lowest levels) of neutrophil counts no less than 1500/mm³, while they are decreased to less than 100/mm³ with the previous methods.

In some embodiments, the hematopoietic chimerism is characterized by a percentage of donor cells in the lymphohematopoietic system of at least 5%, 10%, 15%, 20%, 25%, 30%, 35%, 40%, 45%, 50%, 55%, 60%, 65%, 70%, 75%, 80%, 85%, or at least 90%.

In some embodiments, the hematopoietic chimerism is characterized by a percentage of donor cells in the lymphohematopoietic system of at least 10%.

In some embodiments, the hematopoietic chimerism is transient or persists indefinitely.

In some embodiments, the method is performed such that immune reconstitution is essentially complete, or within the normal reference range, at most 2 months, 3 months, 4 months, 5 months, 6 months, 7 months, 8 months, 9 months, 10 months, 11 months, or at most 1 year after completion of the method. In some embodiments, wherein the method is performed such that immune reconstitution is essentially complete, or within the normal reference range, at most 2 months.

In some embodiments, the inhibitor is administered to the patient for 10 doses of 10 mg/kg, 11 doses of 10 mg/kg, 12 doses of 10 mg/kg, 13 doses of 10 mg/kg, 14 doses of 10 mg/kg, or 15 doses of 10 mg/kg. In some embodiments, the inhibitor is administered to the patient for 11 doses of 10 mg/kg. In some embodiments, the inhibitor is administered to the patient at a daily dose of 10 mg/kg.

In some embodiments, the inhibitor is administered to the patient intravenously, intravascularly, topically, intraarterially, intracranially, intramuscularly, orally, intraorbitally, by inhalation, transdermally, or intraperitonially. In some embodiments, the inhibitor is administered to the patient orally.

In some embodiments, the method further comprises administering 1 Gy, 2 Gy, 3 Gy, 4 Gy, 5 Gy, 6 Gy, 7 Gy, 8 Gy, 9 Gy, 10 Gy, 11 Gy, 12 Gy, 13 Gy, 14 Gy, 15 Gy, 16 Gy, 12 Gy, 18 Gy, 19 Gy, or 20 Gy of local thymic irradiation to the patient for induction of allograft tolerance. In some embodiments, the method further comprises administering 7 Gy of local thymic irradiation to the patient for induction of allograft tolerance.

In some embodiments, the method further comprises administering cyclosporine A to the patient.

In some embodiments, the method further comprises administering anti-thymocyte globulin (“ATG”) to the patient. In some embodiments, the ATG is administered to the patient for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days.

In some embodiments, the ATG is administered to the patient at a daily dose of about 0.5 mg/kg, about 1 mg/kg, about 1.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg. In some embodiments, the ATG is administered to the patient intravenously, intravascularly, intraarterially, intracranially, intramuscularly, intraorbitally, transdermally, or intraperitonially.

In some embodiments, the method further comprises transplanting an organ from an organ-donor to the patient. In some embodiments, the organ-donor and the donor of the bone marrow are the same person or the donor of the bone marrow is a person that is HLA-identical to the organ-donor. In some embodiments, the organ is a heart, intestine, kidney, liver, lung, or a pancreas. In some embodiments, the organ is HLA mismatched.

In one aspect, provided herein is a method for performing a bone marrow transplant, wherein the method comprises administering to the recipient of the bone marrow transplant an inhibitor of Mcl-1.

In some embodiments, the inhibitor of Mcl-1 is a specific inhibitor of Mcl-1.

In some embodiments, the inhibitor of Mcl-1 is administered with an inhibitor of a specific inhibitor of Bcl-2. In some embodiments, the inhibitor of Mcl-1 is administered as a monotherapy.

In some embodiments, the inhibitor of Mcl-1 is obatoclax, A-1210477, AMG176, S64315, S63845, or AZD5991.

In some embodiments, the inhibitor of Mcl-1 is administered at about 10 mg, about 20 mg, about 30 mg, about 40 mg, about 50 mg, about 60 mg, about 70 mg, about 80 mg, about 90 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the method further comprises administering an Mcl-1 inhibitor at a dose sufficient to reduce recipient's hematopoietic stem cell in niches.

Also provided herein is a use of an inhibitor of an anti-apoptotic Bcl-2 family member in a method described herein.

### 5. BRIEF DESCRIPTION OF THE FIGURES

FIG. 1 is a non-limiting schematic showing a non-myeloablative conditioning regimen.

FIG. 2A-B show the percentage of donor lymphocytes (FIG. 2A) and granulocytes (FIG. 2B) days after combined kidney and bone marrow transplantation (CKBMT).

FIG. 3A shows hematopoietic chimerism after CKBMT in group A (TBI 3Gy, no venetoclax) and C. 3/3 Group C recipients developed markedly higher and more prolonged Hematopoietic Chimerism (MC), (FIGS. 3A and B) with excellent lymphocyte depletion (FIG. 3A) but without neutropenia (FIGS. 3B and C) in contrast to Group A recipients (3.0 Gy TBI without ABT-199). Recipients in Group B recipients (TBI 1.5 Gy but no ABT-199) failed to develop MC. Group D (no TBI) failed to develop MC, indicating that minimal TBI is necessary even with ABT-199 for induction of MC. Group C recipients achieved long-term allograft survival (>316, >637, >301 days)) without rejection, while all recipients in Groups B (No ABT-199, TBI 1.5Gy), D (ABT-199+, No TBI), E (ABT-199+, No TI) developed rejection.

FIG. 4 shows BAX expression after combined kidney and bone marrow transplantation in group B and C Bcl-2 inhibition effectively induced apoptosis of T cell subset: Expression of BAX in Group C was consistently higher than those in Group B after treatment of ABT-199, indicating that higher levels of intrinsic apoptosis were induced in CD4 and CD8 T cells by adding ABT-199.

FIG. 5 shows a recovery of recent thymic emigrants (RTEs) in monkeys after transplant that did not undergo thymic irradiation (Group E) as compared to Group C which received the same amount of TBI and ABT-199 but RTEs remained suppressed after transplantation.

FIG. 6 shows the conditioning regimen with venetoclax (ABT-199), comprising total body irradiation (TBI) administered at 1.5 Gy, thymic irradiation (TI) administered at 7 Gy, anti-thymocyte globulin (ATG), and anti-CD154 or belatacept.

FIG. 7A-B compares the effect of navitoclax or venetoclax administration in the conditioning regimen. Bax expression analyzed in T cells, B cells and NK cells after treatment with navitoclax or venetoclax as compared to pre Rx levels (n=3) (FIG. 7A). CD4⁺ and CD8⁺ effector memory T cells (TEM) and platelet counts were analyzed after navitoclax or venetoclax treatment (n=3 in each group) (FIG. 7B).

FIG. 8A-C shows lymphocyte deletion and intrinsic apoptosis. T cells, B cells, and NK cell counts in the peripheral blood were measured by flow cytometry (FIG. 8A). t-SNE map of apoptosis related protein expression of the peripheral lymphocytes in a representative recipient (FIG. 8B). Various apoptosis related protein expression on T cells at 2 weeks (measured by CyTOF) (FIG. 8C).

FIG. 9A-B show hematopoietic chimerism and CBC of subjects in Group A, Group C, and Group E. FIG. 9A shows chimerism in the peripheral blood as measured by flow cytometry. Both lymphoid chimerism and myeloid chimerism were significantly higher in Group D vs. Groups A and C. FIG. 9B shows that white blood cell counts, platelet counts, and HCT were stable in Group D while significant pancytopenia was observed in Group A. ****p<0.0001, ***p<0.0003, **p=0.005.

FIG. 10A-C shows the percent survival after of the animals after renal allograft. FIG. 10A shows the renal allograft survival curve (log rank test). Group D vs. F or G (p<0.03); Group D vs. H (p<0.05); Group E vs. F (p<0.03). Renal allograft biopsy taken on day 800 from a Group D recipient showed no diagnostic abnormality (FIG. 10B). Skin transplantation performed one year after CKBMT showed specific acceptance of the skin graft from the kidney and bone marrow donor (lower left). Two skins from third party animals (two skins right side) were rejected within one week (FIG. 10C).

FIG. 11 shows the effect of the costimulatory blockade on chimerism. Myeloid chimerism was highest in Group D recipients (anti-CD154 mAb) vs. Group E (p=0.0003) and Group F (p<0.0001). No chimerism was detected in Group F (without costimulatory blockade).

FIG. 12A-B illustrates the requirement of thymic irradiation. Myeloid chimerism in Group G (n=3) was significantly (p<0.05) inferior to Group D (n=5) (FIG. 12A). CD4⁺ RTE and CD4⁺ Naïve T cells relative to pre-Tx value (ratio post/pre) were significantly higher (p<0.0001) in Group G (n=3) than Group D (n=5) (FIG. 12B).

FIG. 13 illustrates the drug-only treatment protocol comprising the Mcl-1 inhibitor (S63845) and venetoclax (ABT-199).

FIG. 14 shows the suppression of hematopoietic stem cells (HSCs) and total colony forming units (CFUs) resulting from each of the three drug treatment protocols of FIG. 13.

FIG. 15 illustrates the treatment protocol comprising the Mcl-1 inhibitor (S63845) and venetoclax (ABT-199) administration and the timing of the bone marrow transplant.

FIG. 16 shows suppression of hematopoietic stem cells (HSCs) and total colony forming units (CFUs) after BMT on day 6 by CFU assay and flow cytometry, resulting from each of the three drug treatment protocols of FIG. 15.

FIG. 17A-B shows multilineage hematopoietic chimerism in two of the three bone marrow transplants. FIG. 17A shows the increase of multilineage chimerism starting on day 9 to day 21. FIG. 17B shows lymphocyte chimerism and granulocyte chimerism are also detectable.

### 6. DETAILED DESCRIPTION OF THE INVENTION

Provided herein are methods for inducing hematopoietic chimerism (e.g. mixed chimerism, full donor chimerism) using an inhibitor of an anti-apoptotic Bcl-2 inhibitor. The methods as described herein can be used to induce permanent hematopoietic chimerism (e.g., for recipients of a bone marrow or hematopoietic stem cell (HSC) transplant) or transient chimerism (e.g., for recipients of a solid organ transplant in combination with a bone marrow of HSC infusion). In specific embodiments, the methods provided herein are methods for conducting a combined bone marrow (HSC) and solid organ transplantation using the conditioning regimen and the postoperative regimen described herein. The methods include a conditioning regimen as described in Section 6.4. More specifically, an inhibitor of an anti-apoptotic Bcl-2 can be used with the conditioning regimen. Uses of such inhibitors with the methods, kits, and systems are also provided. Transplantation and bone marrow or HSC infusion are described in Section 6.2. Organs that can be transplanted using the methods provided herein are described in section 6.2. The method includes a postoperative regimen as described in 6.7. Examples of the methods provided herein are described in Section 7.

In one aspect, provided herein are methods for inducing hematopoietic chimerism in a patient in need thereof, wherein the method comprises, administering a specific inhibitor of Bcl-2; and transplanting bone marrow of HSCs from a donor to the patient, such that hematopoietic chimerism is induced in the patient. In certain embodiments, hematopoietic chimerism includes, but is not limited to, mixed chimerism and full donor chimerism.

In some embodiments, a method provided herein comprises inducing hematopoietic chimerism in a patient in need thereof by administering an inhibitor of an anti-apoptotic Bcl-2 family member (e.g., Bcl-2) and total body irradiation (“TBI”) to the patient and infusing bone marrow or HSCs from a donor to the patient. In specific embodiments, the inhibitor of an anti-apoptotic Bcl-2 family member is a specific inhibitor of Bcl-2. In even more specific embodiments, the specific inhibitor of Bcl-2 is venetoclax, oblimersen, PNT2258, or SPC2996. In an even more specific embodiment, venetoclax and a low dose of TBI (e.g., 1.5 Gy) are administered to the patient during the conditioning period prior to infusion of the bone marrow or HSCs from a donor. Such low dose regimens of TBI are described in Section 6.4.2. In certain embodiments, bone marrow from an allogeneic donor is subsequently administered to the recipient. Such conditioning regimens can further be combined with thymic irradiation (“TI”).

In certain embodiments, the method comprises administering during the conditioning regimen a combination of two or more different inhibitors of anti-apoptotic Bcl-2 family members. In a specific embodiment, the combination is a combination of an inhibitor of Bcl-2 (such as venetoclax, oblimersen, PNT2258, or SPC2996) and an inhibitor of Mcl-1 (such as obatoclax, A-1210477, S64315, AMG176, S63845, or AZD5991). In certain embodiments, such a combination of inhibitors is further combined with low dose TBI (as described in Section 6.4.2). In a certain embodiment, the initial administration of the first inhibitor of an anti-apoptotic Bcl-2 family member to the patient occurs at the same time as the initial administration of the second anti-apoptotic BCL-2 family member to the patient. Such conditioning regimens can further be combined with thymic irradiation (“TI”). Without being bound by theory, such an inhibitor of Mcl-1 clears so-called niches of recipient's hematopoietic stem cells. In certain embodiments, addition of the administration of the Mcl-1 inhibitor deletes hematopoietic stem cells in bone marrow niches. In certain embodiments, addition of the administration of the Mcl-1 inhibitor reduces the amount of hematopoietic stem cells in the recipient's bone marrow by at least 10%, 15%, 20%, 25%, 30%, 35%, 40%, 45%, 50%, 55%, 60%, 65%, 70%, 75%, 80%, 85%, 90%, or 95% as compared to pre-treatment levels.

In certain embodiments, the method comprises administering during the conditioning regimen a monotherapy comprising a Mcl-1 inhibitor (Mcl-1i). The Mcl-1i can be, but is not limited to, obatoclax, A-1210477, S64315, AMG176, S63845, or AZD5991. In certain embodiments, such a monotherapy of Mcl-1i is further combined with low dose TBI (as described in Section 6.4.2). Such a conditioning regimen can further be combined with thymic irradiation (“TI”). Without being bound by theory, such an inhibitor of Mcl-1 clears so-called niches of recipient's hematopoietic stem cells. In certain embodiments, administration of the Mcl-1 inhibitor deletes hematopoietic stem cells in bone marrow niches. In certain embodiments, administration of the Mcl-1 inhibitor reduces the amount of hematopoietic stem cells in the recipient's bone marrow by at least 10%, 15%, 20%, 25%, 30%, 35%, 40%, 45%, 50%, 55%, 60%, 65%, 70%, 75%, 80%, 85%, 90%, or 95% as compared to pre-treatment levels.

In certain embodiments, the deletion of CD34⁺ cells of the bone marrow can be determined using flow cytometry and comparing the amount of CD34⁺ cells of the bone marrow to pre-treatment levels. The reduction in total colony forming units can be measured by a CFU assay and comparing the CFUs to pre-treatment levels.

In one aspect, provided herein are methods for inducing hematopoietic chimerism in a patient in need thereof, wherein the method comprises, administering a first course of a first inhibitor of an anti-apoptotic Bcl-2 family member to the patient, transplanting bone marrow from a donor to the patient, and administering a second course of a second inhibitor of an anti-apoptotic Bcl-2 family member to the patient starting 1, 2, 3, 4, 5, 6, 7, 8, 9, or 10 days after completion of the first course, such that hematopoietic chimerism is induced in the patient. In certain embodiments, the second course is initiated, 1, 2, 3, 4, 5, 6, 7, 8, 9, or 10 days after the bone marrow transplant. In a specific embodiment, the Bcl-2 inhibitor “holiday” can be 2, 3, or 4 days long. In an even more specific embodiment, the second course of a second inhibitor is given starting 3 days after completion of the first course. Bcl-2 inhibitors that can be used during the first course and the second course are described in 6.4.1. Without being bound by theory, such treatment with two courses reduces the risk of and/or severity of graft-versus-host disease.

In one aspect, provided herein are methods for inducing hematopoietic chimerism in a patient in need thereof, wherein the method comprises, administering an inhibitor of an anti-apoptotic Bcl-2 family member to the patient, administering TBI to the patient; and transplanting bone marrow (HSCs) and an organ selected from heart, intestine, kidney, or liver from a donor to the patient.

In one aspect, provided herein are methods for performing a bone marrow (HSC) transplant, wherein the method comprises administering to the recipient of the bone marrow (HSC) transplant an inhibitor of Mcl-1.

In certain embodiments, provided herein are methods for inducing hematopoietic chimerism in a patient in need thereof, wherein the method comprises administering an inhibitor of an anti-apoptotic Bcl-2 family member to the patient, wherein the patient is not being treated with an anti-CD154 antibody. Also provided herein are methods for performing an organ transplantation of an organ described in Section 6.2 using a conditioning regimen that comprises administration of an inhibitor of an anti-apoptotic Bcl-2 family member (see Section 6.4.1), followed by bone marrow or HSC transfusion and organ transplantation as described in Sections 6.5 and 6.6, followed by post-operative treatment as described in Section 6.7, wherein the patient (the recipient of the organ transplant) is not receiving treatment with an inhibitor of CD154 (such as an anti-CD154 antibody).

In certain embodiments, induced hematopoietic chimerism in the patient in need thereof can persist indefinitely after the bone marrow transplantation. In certain embodiments, hematopoietic chimerism can be transient such as in the context of transplantation of a solid organ. In certain embodiments, the methods induce tolerance in the recipient towards the transplanted organ.

In certain embodiments, the methods provided herein results in hematopoietic chimerism without neutropenia or with reduced neutropenia. For example, the methods provided herein that comprise administration of an inhibitor of an anti-apoptotic member of the Bcl-2 protein family results in at least 5%, 10%, 15%, 20%, 25%, 30%, 35%, 40%, 45%, 50%, 55%, 60%, 65%, 70%, 75%, 80%, 85%, 90%, 95%, or even 100% less neutropenia as compared to the same treatment regimen in the absence of administration of an inhibitor of an anti-apoptotic member of the Bcl-2 protein family.

In some embodiment, chimerism can be induced without need of myelosuppressive treatments. In a specific embodiment, chimerism can be induced without a need of TBI. In a specific embodiment, chimerism can be induced without a need of cyclophosphamide. In some embodiments, the conditioning regimen can result in no presence of donor-derived antigen-presenting cells. In some embodiments, the conditioning regimen can result in removal of peripheral donor cells such as, reactive lymphocytes. In some embodiments, the conditioning regimen improves immunological tolerance. In some embodiments, the conditioning regimen promotes T-cell tolerization. In some embodiments, the conditioning regimen can prevent myelosuppression. In some embodiments, administering an inhibitor of an anti-apoptotic Bcl-2 family member (e.g., Bcl-2) can induce apoptosis in donor-reactive T-cells. In some embodiments, administering an inhibitor of an anti-apoptotic Bcl-2 family member (e.g., Bcl-2) can inhibit allogeneic immune responses.

In some embodiments, the conditioning regimen can result in no presence of donor-derived antigen-presenting cells. In some embodiments, the conditioning regimen can result in removal of peripheral donor cells such as, reactive lymphocytes. In some embodiments, the conditioning regimen can improve immunological tolerance. In some embodiments, the conditioning regimen can prevent myelosuppression.

In some embodiments, the conditioning regimen reduces the risk or severity of Graft versus Host Disease (GVHD) in the recipient. In some embodiments, the conditioning regimen can result in allograft tolerance.

### 6.1 Recipients

Individuals who have one organ, or more than one organ, that has been damaged by means including injury, disease, or birth defect may meet the criteria to receive an organ transplant. Individuals who have been selected to receive an organ transplant may follow these methods described herein with the goal of inducing a state of hematopoietic chimerism (e.g. mixed chimerism, full donor chimerism) in which the recipient and donor hematopoietic cells coexist in the recipient. When the state of hematopoietic chimerism is achieved, it reduces the need for long-term immunosuppressive therapies. The recipient can be HLA-matched or HLA-mismatched with the donor. In certain embodiments, the recipient is a human.

In other embodiment, the methods herein provide for the transplantation of bone marrow or HSCs. In those situations, hematopoietic chimerism can be permanent. In certain embodiments, hematopoietic chimerism includes, but is not limited to, mixed chimerism and full donor chimerism.

### 6.2 Organ Transplant

As used herein, the donor is the individual from which the organ to be transplanted is taken. The donor is of the same species as the recipient and the donor can be alive or deceased. The donor can be related to the recipient or not related to the recipient. As used herein, the recipient is the individual that will receive the transplanted organ. The recipient can be related or not related to the donor. The donor can be HLA-matched or HLA-mismatched with the recipient.

Organs that can be transplanted utilizing the methods provided herein can be any solid organ. In some embodiments, the organ can be a kidney, heart, intestine, liver, lung, pancreas or other organ that can be transplanted using the methods provided herein. In some embodiments, the organ can be a vascular-composite allograft including hands, feet, other limbs, faces, or other body parts that can be transplanted using the methods provided herein. In some embodiments, the transplanted organ may be whole organ, a part of an organ, or cells derived from an organ.

In a specific embodiment, the organ that can be transplanted is a heart, intestine, kidney, liver, lung, or a pancreas. In some embodiment, the organ that can be transplanted is not skin.

### 6.3 Bone Marrow (Hematopoietic Stem Cells)

Hematopoietic stem cells (HSCs) can be derived from bone marrow of the donor or obtained by leukapheresis. In some embodiments, the HSCs can be obtained by methods including, but not limited to, aspirated bone marrow cells (e.g. from live donor), isolated from bones (e.g. from cadaver donor), or isolating peripheral blood stem cells (PBSC) from live donors (e.g. by leukapheresis). In some embodiments, HSCs, obtained from the organ donor, can be HLA-matched to the recipient. In certain embodiments, HSCs, obtained from the organ donor, can be HLA-mismatched. In certain embodiments, the bone HSC transplant can be combined with the organ transplant, can occur after the organ transplant, or can occur prior to the organ transplant. In certain embodiments, the organ donor and the HSCs are the same person; in other embodiments, the organ donor and the HSCs are different.

In certain embodiments, the desired outcome is to induce a state of chimerism in the recipient. By performing a combined transplant of a solid organ and an infusion of HSCs from unprocessed donor bone marrow or leukapheresis, in combination administering an inhibitor of an anti-apoptotic Bcl-2 family member (e.g., Bcl-2), the recipient can develop hematopoietic chimerism, which in turn can result in immune tolerance towards the organ by the recipient's immune system. In certain embodiments, the desired outcome allows for the reduction of the amount of TBI necessary to achieve immune tolerance towards the organ. In certain embodiments, the dosage of TBI administered to a recipient is at most 1.5 Gy, 1.4 Gy, 1.3 Gy, 1.2 Gy, 1.1 Gy, 1.0 Gy, 0.9 Gy, 0.8 Gy, 0.7 Gy, 0.6 Gy, 0.5 Gy, 0.4 Gy, 0.3 Gy, 0.2 Gy, or 0.1 Gy. In certain embodiments, the desired outcome allows for the elimination of the TBI requirement in order to achieve immune tolerance towards the organ.

As used herein, “chimerism” refers to a state where donor hematopoietic cells exist in the recipient blood. In certain embodiments, the recipient may be monitored to assess the presence of hematopoietic chimerism. In certain embodiments, the recipient will have at least 1% circulating donor hematopoietic cells. In certain embodiments, the recipient will have at least 5%, 10%, 15%, 20%, 25%, 30%, 35%, 40%, 45%, 50%, 55%, 60%, 65%, 70%, 75%, 80%, 85%, 90%, or 95% circulating donor hematopoietic cells. In certain embodiments, the recipient will have 1-10%, 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, 80-90%, or 85-95% circulating donor hematopoietic cells. In certain embodiments, induced chimerism can last for hematopoietic and/or immune cells, for a period of time, for example for 1 month, 2 months, 3 months, 4 months, 5 months, or 6 months. In a specific embodiment, the chimerism will persist in the recipient for at least 6 months.

In some embodiments, induced hematopoietic chimerism persists indefinitely. In certain embodiments, hematopoietic chimerism includes, but is not limited to, mixed chimerism and full donor chimerism.

### 6.4 Conditioning Regimen

The conditioning regimen or use with the methods provided herein can include administration of one or more of the treatments below. In some embodiments, the conditioning regimen to be used with the methods provided herein does not include administration of an anti-CD154 treatment, such as administration of an antibody that specifically binds to CD154.

### 6.4.1 Bcl-2 Inhibitor Treatment

In some embodiments, an inhibitor of an anti-apoptotic Bcl-2 family member as described herein can be, but is not limited to, a small molecule, a peptide, polypeptide, protein, fusion protein, an antibody, an antisense oligonucleotide, RNAi, or other modalities that suppress the expression of the target gene.

In some embodiments, the conditioning regimen can include administering an inhibitor of an anti-apoptotic Bcl-2 family member to the patient. Such an inhibitor can be specific to a particular anti-apoptotic Bcl-2 family member, and reduces or eliminates the activity or levels of that specific protein. In other embodiments, an inhibitor of an anti-apoptotic Bcl-2 family member can have a broader target spectrum such that it inhibits the activity or levels of two or more anti-apoptotic Bcl-2 family members.

In certain embodiments, an inhibitor of an anti-apoptotic Bcl-2 family member is specific to the founding member of the Bcl-2 family, namely Bcl-2. In an even more specific embodiment, the inhibitor inhibits the activity of Bcl-2. In certain embodiments, the inhibitor of Bcl-2 is venetoclax, oblimersen, PNT2258, or SPC2996.

In certain embodiments, the conditioning regimen can include administering an inhibitor of an anti-apoptotic BCL-2 family member, which is a combination of inhibitors of Mcl-1 and Bcl-2. In a certain embodiments, the inhibitor of Mcl-1 administered in combination with Bcl-2 is obatoclax, A-1210477, AMG176, S64315, S63845, or AZD5991. In a specific embodiment, the inhibitor of Mcl-1 administered in the conditioning regimen is S64315 or S63845.

In some embodiments, an inhibitor of an anti-apoptotic Bcl-2 family member described herein is venetoclax, oblimersen, PNT2258, or SPC2996. In some embodiments, an inhibitor of Mcl-1 described herein is obatoclax, A-1210477, AMG176, S64315, S63845, or AZD5991.

In some embodiments, venetoclax can be administered at 10 mg, about 20 mg, about 30 mg, about 40 mg, about 50 mg, about 60 mg, about 70 mg, about 80 mg, about 90 mg, about 100 mg, about 150 mg, about 200 mg, about 300 mg, about 400 mg, about 500 mg, about 600 mg, about 700 mg, about 800 mg, about 900 mg, or about 1000 mg. In a specific embodiment, venetoclax can be administered at about 10 mg, 50 mg, or 100 mg. In a specific embodiment, venetoclax is administered at about 400 mg. In some embodiments, venetoclax can be administered for 1 week, 2 weeks, 3 weeks, 4 weeks, 5 weeks, 6 weeks, 8 weeks, or 12 weeks. In some embodiments, venetoclax can be administered as a single dose. In some embodiments, venetoclax can be administered as 1, 2, 3, 4, 5, 6, 7, 8, 9, 10, 11, 12, 13, 14, 15, 16, 17, 18, 19, 20 unit doses. In some embodiments, venetoclax can be administered the patient for 1 of 10 mg/kg, 2 of 10 mg/kg, 3 of 10 mg/kg, 4 of 10 mg/kg, 5 of 10 mg/kg, 6 of 10 mg/kg, 7 of 10 mg/kg, 8 of 10 mg/kg, 9 of 10 mg/kg, 10 doses of 10 mg/kg, 11 doses of 10 mg/kg, 12 doses of 10 mg/kg, 13 doses of 10 mg/kg, 14 doses of 10 mg/kg, or 15 doses of 10 mg/kg. In a specific embodiment, venetoclax is administered at 10 mg/kg. In some embodiments, venetoclax is administered orally.

In some embodiments, oblimersen can be administered at about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, or 1.5 mg/kg, 2 mg/kg, 2.5 mg/kg, 3 mg/kg, 3.5 mg/kg, 4 mg/kg, 4.5 mg/kg, 5 mg/kg. In a specific embodiment, oblimersen can be administered at about 1.5 mg/kg. In some embodiments, oblimersen can be administered as 1, 2, 3, 4, 5, 6, 7, 8, 9, 10, 11, 12, 13, 14, 15, 16, 17, 18, 19, 20 unit doses. In some embodiments, oblimersen can be administered for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days. In some embodiments, oblimersen can be administered for 7 consecutive days. In some embodiments, oblimersen is administered intravenously.

In some embodiments, PNT2258 can be administered at about 10 mg, about 20 mg, about 30 mg, about 40 mg, about 50 mg, about 60 mg, about 70 mg, about 80 mg, about 90 mg, about 100 mg, about 150 mg, about 200 mg, about 300 mg, about 400 mg, or about 500 mg. In a specific embodiment, PNT2258 can be administered at about 120 mg. In some embodiments, PNT2258 can be administered for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days. In some embodiments, PNT2258 can be administered orally.

In some embodiments, SPC2996 can be administered at 10 mg, about 20 mg, about 30 mg, about 40 mg, about 50 mg, about 60 mg, about 70 mg, about 80 mg, about 90 mg, or about 100 mg. In a specific embodiment, SPC2996 can be administered at about 50 mg. In some embodiments, SPC2996 can be administered intravenously. In some embodiments, SPC2996 is administered for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days.

In some embodiments, A-1210477 can be administered at 10 mg, about 20 mg, about 30 mg, about 40 mg, about 50 mg, about 60 mg, about 70 mg, about 80 mg, about 90 mg, about 100 mg, about 150 mg, about 200 mg, about 300 mg, about 400 mg, or about 500 mg.

In some embodiments, AMG176 can be administered at 10 mg, about 20 mg, about 30 mg, about 40 mg, about 50 mg, about 60 mg, about 70 mg, about 80 mg, about 90 mg, about 100 mg, about 150 mg, about 200 mg, about 300 mg, about 400 mg, or about 500 mg.

In some embodiments, S64315 can be administered at about 10 mg, about 20 mg, about 30 mg, about 40 mg, about 50 mg, about 60 mg, about 70 mg, about 80 mg, about 90 mg, about or 100 mg. In some embodiments, S64315 can be administered at about 50 mg. In some embodiments, S64315 for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days. In some embodiments, S64315 is administered for 1 week, 2 weeks, 3 weeks, 4 weeks, 5 weeks, 6 weeks, 8 weeks, or 12 weeks. In some embodiments, S64315 can be administered once a week.

In some embodiments, S63845 can be administered at about 10 mg, about 20 mg, about 30 mg, about 40 mg, about 50 mg, about 60 mg, about 70 mg, about 80 mg, about 90 mg, about 100 mg, about 150 mg, about 200 mg, about 300 mg, about 400 mg, or about 500 mg. In some embodiments, S63845 can be administered at about 50 mg. In some embodiments, S63845 can be administered for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days. In some embodiments, S63845 is administered for 1 week, 2 weeks, 3 weeks, 4 weeks, 5 weeks, 6 weeks, 8 weeks, or 12 weeks. In some embodiments, S63845 can be administered once a week. In some embodiments, S63845 is administered intravenously.

In some embodiments, AZD5991 can be administered at about 10 mg, about 20 mg, about 30 mg, about 40 mg, about 50 mg, about 60 mg, about 70 mg, about 80 mg, about 90 mg, about 100 mg, about 150 mg, about 200 mg, about 300 mg, about 400 mg, or about 500 mg. In some embodiments, AZD5991 can be administered for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days. In some embodiments, AZD5991 is administered intravenously.

In some embodiments, an inhibitor of an anti-apoptotic Bcl-2 family member described herein can be administered at a dosage in the range of 0.1 mg/kg to about 1 mg/kg, about 0.1 mg/kg to about 1.5 mg/kg, about 0.1 mg/kg to about 2 mg/kg, about 0.1 mg/kg to about 2.5 mg/kg, about 0.1 mg/kg to about 3 mg/kg, about 0.1 mg/kg to about 3.5 mg/kg, about 0.1 mg/kg to about 4 mg/kg, about 0.1 mg/kg to about 4.5 mg/kg, about 0.1 mg/kg to about 5 mg/kg, about 0.1 mg/kg to about 5.5 mg/kg, about 0.1 mg/kg to about 6 mg/kg, about 0.1 mg/kg to about 6.5 mg/kg, about 0.1 mg/kg to about 7 mg/kg, about 0.1 mg/kg to about 7.5 mg/kg, about 0.1 mg/kg to about 8 mg/kg, about 0.1 mg/kg to about 8.5 mg/kg, about 0.1 mg/kg to about 9 mg/kg, about 0.1 mg/kg to about 9.5 mg/kg, about 0.1 mg/kg to about 10 mg/kg, about 0.1 mg/kg to 50 mg/kg, or about 1 mg/kg to 50 mg/kg, of the human subject's body weight.

In a specific embodiment, the inhibitor of an anti-apoptotic Bcl-2 family member described herein can be administered at a dosage of 10 mg/kg.

In a specific embodiment, an inhibitor of an anti-apoptotic Bcl-2 family member described herein can be administered at a dosage of 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg of the human subjects body weight.

In a specific embodiment, the inhibitor of an anti-apoptotic Bcl-2 family member described herein can be administered at a dosage of 10 mg/kg.

In certain embodiments, the specific inhibitor of Bcl-2 is administered to the patient for 1 of 10 mg/kg, 2 of 10 mg/kg, 3 of 10 mg/kg, 4 of 10 mg/kg, 5 of 10 mg/kg, 6 of 10 mg/kg, 7 of 10 mg/kg, 8 of 10 mg/kg, 9 of 10 mg/kg, 10 doses of 10 mg/kg, 11 doses of 10 mg/kg, 12 doses of 10 mg/kg, 13 doses of 10 mg/kg, 14 doses of 10 mg/kg, or 15 doses of 10 mg/kg.

In some embodiments, an inhibitor of an anti-apoptotic Bcl-2 family member described herein can be administered at unit dose of 0.1 mg to 1000 mg or 1 mg to 500 mg. In certain embodiments, an inhibitor of an anti-apoptotic Bcl-2 family member can be administered at unit dose of about 0.1 mg to 1900 mg, 0.1 mg to 1800 mg, 0.1 mg to 1700 mg, 0.1 mg to 1600 mg, 0.1 mg to 1500 mg, 0.1 mg to 1400 mg, 0.1 mg to 1300 mg, 0.1 mg to 1200 mg, 0.1 mg to 1000 mg, 0.1 mg to 900 mg, 0.1 mg to 800 mg, 0.1 mg to 700 mg, 0.1 mg to 600 mg, 0.1 mg to 500 mg, 0.1 mg to 400 mg, 0.1 mg to 300 mg, 0.1 mg to 200 mg, 0.1 mg to 100 mg.

In some embodiments, an inhibitor of an anti-apoptotic Bcl-2 family member described herein can be administered as single dose.

In some embodiments, an inhibitor of an anti-apoptotic Bcl-2 family member described herein can be administered as 1, 2, 3, 4, 5, 6, 7, 8, 9, 10, 11, 12, 13, 14, 15, 16, 17, 18, 19, 20 unit doses. In some embodiments, an inhibitor of an anti-apoptotic Bcl-2 family member described herein can be administered as 1, 2, 3, 4, 5, 6, 7, 8, 9, 10, 11, 12, 13, 14, 15, 16, 17, 18, 19, 20 unit doses of about 10 mg/kg.

In some embodiments, an inhibitor of an anti-apoptotic Bcl-2 family member described herein can be administered for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days.

In some embodiments, an inhibitor of an anti-apoptotic Bcl-2 family member described herein can be administered once per every two weeks or once a month or once every 3 to 6 months for a period of one year or over several years, or over several year-intervals. In some embodiments, an inhibitor of an anti-apoptotic Bcl-2 family member described herein can be administered every 2 weeks, every 4 weeks, every 6 weeks, every 8 weeks, every 10 weeks, or every 12 weeks.

In some embodiments, an inhibitor of an anti-apoptotic Bcl-2 family member described herein can be administered weekly, monthly, every 3 months, every 6 months or yearly. In some embodiments, an inhibitor of an anti-apoptotic Bcl-2 family member described herein can be administered for 1 week, 2 weeks, 3 weeks, 4 weeks, 5 weeks, 6 weeks, 8 weeks, or 12 weeks. In some embodiments, an inhibitor of an anti-apoptotic Bcl-2 family member described herein can be administered for 1 month, 2 months, 3 months, 4 months, 5 months, 6 months, 7 months, 8 months, 9 months, 10 months, 11 months, 12 months, 15 months, 18 months, or 24 months.

In some embodiments, an inhibitor of an anti-apoptotic Bcl-2 family member described herein may be an antisense oligonucleotide. Antisense oligonucleotides can comprise peptide nucleic acids (PNAs), which contain a peptide-based backbone rather than a sugar-phosphate backbone. Other modified sugar or phosphodiester modifications to the antisense oligonucleotide are also contemplated. By way of non-limiting examples, other chemical modifications can include 2′-O-alkyl (e.g., 2′-O-methyl, 2′-O-methoxyethyl), 2′-fluoro, and 4′-thio modifications, and backbone modifications, such as one or more phosphorothioate, morpholino, or phosphonocarboxylate linkages. In some embodiments, an inhibitor of an anti-apoptotic Bcl-2 family member described herein may be an antisense oligonucleotide. In some embodiments, an inhibitor of an anti-apoptotic Bcl-2 family member described herein may be an antisense oligonucleotide, which targets all the members of anti-apoptotic Bcl-2 family. In some embodiments, an inhibitor of an anti-apoptotic Bcl-2 family member described herein may be an antisense oligonucleotide, which targets specific members of anti-apoptotic Bcl-2 family.

In some embodiments, an inhibitor of an anti-apoptotic Bcl-2 family member described herein may be a small molecule or a chemical compound. In some embodiments, an inhibitor of an anti-apoptotic Bcl-2 family member described herein may be a small molecule, which targets all the members of anti-apoptotic Bcl-2 family. In some embodiments, an inhibitor of an anti-apoptotic Bcl-2 family member described herein may be a small molecule or a chemical compound, which targets specific members of anti-apoptotic Bcl-2 family.

In some embodiments, an inhibitor of an anti-apoptotic Bcl-2 family member described herein may be an antibody or antigen-fragment thereof. In some embodiments, an inhibitor of an anti-apoptotic Bcl-2 family member described herein may be an antibody or antigen-fragment thereof, which targets all the members of anti-apoptotic Bcl-2 family. In some embodiments, an inhibitor of an anti-apoptotic Bcl-2 family member described herein may be an antibody or antigen-fragment thereof, which targets specific members of anti-apoptotic Bcl-2 family.

As used herein “specific” and/or “specific inhibitor” refers to significant more potency for a respective target when compared to other members of the same class.

### 6.4.2 Total Body Irradiation

In certain embodiments, the conditioning regimen provided herein comprises the recipient undergoing total body irradiation before the transplant surgery. In some embodiments, administering an inhibitor of Bcl-2 to the patient allows for a decreased and/or reduced dosage of TBI. In certain embodiments, the conditioning regimen can include inducing hematopoietic chimerism in a patient by administering a low dose of TBI to the patient. In a specific embodiment, TBI is administered at a very low dose. In some embodiments, TBI is administered at 0.1 Gy, 0.2 Gy, 0.3 Gy, 0.4 Gy, 0.5 Gy, 1 Gy, 1.5 Gy, 2 Gy, 2.5 Gy, or 3 Gy.

In some embodiments, the total body irradiation can be performed 3 days before the transplant, 4 days before the transplant, 5 days before the transplant, 6 days before the transplant, 7 days before the transplant, 3 and 4 days before the transplant, 3 and 5 days before the transplant, 4 and 5 days before the transplant, 4 and 6 days before the transplant, 5 and 6 days before the transplant, 5 and 7 days before the transplant, or 6 and 7 days before the transplant surgery.

In certain embodiments, the dosage of TBI is at least 0.1 Gy, 0.2 Gy Gy, 0.3 Gy, 0.4 Gy, 0.5 Gy, 0.6 Gy, 0.7 Gy, 0.8 Gy, 0.9 Gy, 1 Gy, 1.1 Gy, 1.2 Gy, 1.3 Gy, 1.4 Gy, 1.5 Gy, 1.6 Gy, 1.7 Gy, 1.8 Gy, 1.9 Gy, 2 Gy, 2.1 Gy, 2.2 Gy, 2.3 Gy, 2.4 Gy, 2.5 Gy, 2.6 Gy, 2.7 Gy, 2.8 Gy, 2.9 Gy, or 3 Gy. In certain embodiments, the dosage of TBI is at least 0.1 Gy, 0.2 Gy, 0.3 Gy, 0.4 Gy, 0.5 Gy, 0.75 Gy, 1.0 Gy, 1.25 Gy, 1.50 Gy, 1.75 Gy, 2.0 Gy, 2.25 Gy, 2.50 Gy, 0.5-1.5 Gy, 0.75-1.25 Gy, 1.0-2.0 Gy, 1.25-2.25 Gy, or 1.50-2.50 Gy. In certain embodiments, the dosage of TBI is at least 0.1 Gy to 0.5 Gy, 0.1 Gy to 1 Gy, 0.1 Gy to 2 Gy, 0.1 Gy to 2.5 Gy or 0.1 Gy to 3 Gy, inclusive of the endpoints. In certain embodiments, the dosage of total body irradiation is at least 0.1 to 2.5 Gy. In a specific embodiment, the dosage of total body irradiation is at least 1.5 Gy.

In certain embodiments, the dosage of TBI is at most 0.1 Gy, 0.2 Gy Gy, 0.3 Gy, 0.4 Gy, 0.5 Gy, 0.6 Gy, 0.7 Gy, 0.8 Gy, 0.9 Gy, 1 Gy, 1.1 Gy, 1.2 Gy, 1.3 Gy, 1.4 Gy, 1.5 Gy, 1.6 Gy, 1.7 Gy, 1.8 Gy, 1.9 Gy, 2 Gy, 2.1 Gy, 2.2 Gy, 2.3 Gy, 2.4 Gy, 2.5 Gy, 2.6 Gy, 2.7 Gy, 2.8 Gy, 2.9 Gy, or 3 Gy. In certain embodiments, the dosage of TBI is at least 0.1 Gy, 0.2 Gy, 0.3 Gy, 0.4 Gy, 0.5 Gy, 0.75 Gy, 1.0 Gy, 1.25 Gy, 1.50 Gy, 1.75 Gy, 2.0 Gy, 2.25 Gy, 2.50 Gy, 0.5-1.5 Gy, 0.75-1.25 Gy, 1.0-2.0 Gy, 1.25-2.25 Gy, or 1.50-2.50 Gy. In certain embodiments, the dosage of TBI is at least 0.1 Gy to 0.5 Gy, 0.1 Gy to 1 Gy, 0.1 Gy to 2 Gy, 0.1 Gy to 2.5 Gy or 0.1 Gy to 3 Gy, inclusive of the endpoints. In certain embodiments, the dosage of total body irradiation is at least 0.1 to 2.5 Gy. In a specific embodiment, the dosage of total body irradiation is at least 1.5 Gy.

In certain embodiments, the recipient can undergo a single dose of TBI. In certain embodiments, the recipient can undergo two fractionated doses of TBI in two days. In a specific embodiment, the recipient can undergo a TBI of 1.5 Gy, in two consecutive days, 5 days and 4 days before the transplant surgery. In a specific embodiment, the recipient can undergo a total body irradiation of 1.5 Gy in two consecutive days, 6 days and 5 days before the transplant surgery.

### 6.4.3 Thymic Irradiation

In certain embodiments, the conditioning regimen provided herein comprises the recipient undergoing thymic irradiation before the transplant surgery. In some embodiments, the thymic irradiation can be performed 1 day before, 2 days before, or 1 and 2 days before the transplant surgery. In certain embodiments, the dosage of thymic irradiation was such as to be sufficient to deplete intrathymic T-cells. In some embodiments, the thymic radiation is administered locally.

In certain embodiments, the dosage of thymic irradiation can be 100-1000 cGy (centigray). In certain embodiments, the dosage of thymic irradiation can be 1 Gy, 2 Gy, 3 Gy, 4 Gy, 5 Gy, 6 Gy, 7 Gy, 8 Gy, 9 Gy, 10 Gy, 11 Gy, 12 Gy, 13 Gy, 14 Gy, 15 Gy, 16 Gy, 12 Gy, 18 Gy, 19 Gy, or 20 Gy. In a specific embodiment, the recipient can undergo a thymic irradiation of 7 Gy on the day before the transplant surgery.

In certain embodiments, the dosage of thymic irradiation is sufficient to reduce the level of recent thymic emigrants by 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% compared to the level of recent thymic emigrants before thymic irradiation.

### 6.4.4 Anti-Thymocyte Globulin

In some embodiments, the conditioning regimen further comprises optionally administering an Anti-thymocyte globulin (“ATG”) to the patient. In some embodiments, the ATG comprises THYMOGLOBULIN® or Atgam®. In some embodiments, the ATG is administered to the patient for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days.

In some embodiments, the ATG is administered to the patient at a daily dose of about 0.5 mg/kg, about 1 mg/kg, about 1.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg. In some embodiments, the ATG is administered to the patient intravenously, intravascularly, intraarterially, intracranially, intramuscularly, intraorbitally, transdermally, or intraperitonially.

### 6.4.5 Conditioning Regimen Comprising a Bcl-2 Inhibitor and an Mcl-1 Inhibitor

In certain embodiments, hematopoietic chimerism (e.g. mixed chimerism, full donor chimerism) can be induced in a patient in need thereof by administering a first inhibitor of an anti-apoptotic Bcl-2 family member to the patient and a second inhibitor of an anti-apoptotic Bcl-2 family member to the patient. In certain embodiments, the first inhibitor of an anti-apoptotic Bcl-2 family member is a Bcl-2 inhibitor. In certain embodiments, the second inhibitor of an anti-apoptotic Bcl-2 family member is an Mcl-1 inhibitor. The inhibitors are administered to the patient such that hematopoietic chimerism is induced in the patient. In a certain embodiment, administration of a Bcl-2 inhibitor and an Mcl-1 inhibitor comprise a treatment regimen to induce hematopoietic chimerism in the recipient. In certain embodiments, the initial administration of a Bcl-2 inhibitor to the recipient occurs at most 1 day, 2 days, 3 days 4 days, or 5 days before the initial administration of an Mcl-1 inhibitor to the recipient. In certain embodiments, the initial administration of an Mcl-1 inhibitor to the recipient occurs at most 1 day, 2 days, 3 days 4 days, or 5 days before the initial administration of a Bcl-2 inhibitor to the recipient. In a specific embodiments, the initial administration of a Bcl-2 inhibitor to the recipient occurs at the same time as the initial administration of an Mcl-1 inhibitor to the recipient. In a specific embodiment, a Bcl-2 inhibitor and an Mcl-1 inhibitor are administered such to achieve sustained chimerism in a recipient.

In a certain embodiment, an inhibitor of Bcl-2 may be administered in a treatment regimen also comprising an Mcl-1 inhibitor. In certain embodiments, a Bcl-2 inhibitor may be administered to the patient beginning 8 days before transplant, beginning 7 days before transplant, beginning 6 days before transplant, beginning 5 days before transplant, beginning 4 days before transplant, beginning 3 days before transplant, beginning 2 days before transplant, beginning 1 day before transplant, or beginning the day of transplant. In certain embodiments, a Bcl-2 inhibitor is administered daily. In certain embodiments, a Bcl-2 inhibitor is administered more than once per day. In a specific embodiment, administration of a Bcl-2 inhibitor may be administered to the patient beginning 6 days before transplant. In certain embodiments, an inhibitor of Bcl-2 may be administered for a time course of 18 days, 17 days, 16 days, 15 days, 14 days, 13 days, 12 days, 11 days, 10 days, 9 days, 8 days, 7 days, 6 days, 5 days, 4 days, 3 days, 2 days, or 1 day. In a specific embodiment, an inhibitor of Bcl-2 can be administered for 13 days. In a specific embodiment, an inhibitor of Bcl-2 can be administered to a patient for a time course of 13 days beginning 6 days before the transplant.

In certain embodiments, a Bcl-2 inhibitor can administered at a dosage of 15.0 mg/kg, 14.0 mg/kg, 13.0 mg/kg, 12.0 mg/kg, 11.5 mg/kg, 11.0 mg/kg, 10.5 mg/kg, 10.0 mg/kg, 9.5 mg/kg, 9.0 mg/kg, 8.5 mg/kg, 8.0 mg/kg, 7.0 mg/kg, 6.0 mg/kg, 5.0 mg/kg, 4.0 mg/kg, or 3.0 mg/kg. In certain embodiments, a Bcl-2 inhibitor can be administered at a dosage range of 15.0 mg/kg to 13.0 mg/kg, 14.0 mg/kg to 12.0 mg/kg, 13.0 mg/kg to 11.0 mg/kg, 12.0 mg/kg to 10.0 mg/kg, 11.0 mg/kg to 9.0 mg/kg, 10.0 mg/kg to 8.0 mg/kg, 9.0 mg/kg to 7.0 mg/kg, 8.0 mg/kg to 6.0 mg/kg, 7.0 mg/kg to 5.0 mg/kg, 6.0 mg/kg to 4.0 mg/kg, or 5.0 mg/kg to 3.0 mg/kg. In a specific embodiment, a Bcl-2 inhibitor is administered at a dosage amount of 10.0 mg/kg. In certain embodiments, the same dosage amount is administered each day. In certain embodiments, the dosage amount administered is not the same.

In a certain embodiment, an inhibitor of Mcl-1 may be administered in a treatment regimen comprising a Bcl-2 inhibitor. In certain embodiments, an Mcl-1 inhibitor may be administered to the patient beginning 8 days before transplant, beginning 7 days before transplant, beginning 6 days before transplant, beginning 5 days before transplant, beginning 4 days before transplant, beginning 3 days before transplant, beginning 2 days before transplant, beginning 1 day before transplant, or beginning the day of transplant. In certain embodiments, an Mcl-1 inhibitor is administered daily. In certain embodiments, an Mcl-1 inhibitor is administered more than once per day. In a specific embodiment, administration of an Mcl-1 inhibitor may be administered to the patient beginning 6 days before transplant. In certain embodiments, an inhibitor of Mcl-1 may be administered for a time course of 8 days, 7 days, 6 days, 5 days, 4 days, 3 days, 2 days, or 1 day. In a specific embodiment, an inhibitor of Mcl-1 can be administered for 5 days. In a specific embodiment, an inhibitor of Mcl-1 can be administered to a patient for a time course of 5 days beginning 6 days before the transplant. In certain embodiments, an Mcl-1 inhibitor can administered at a dosage of 10 mg/kg, 9.5 mg/kg, 9.0 mg/kg, 8.5 mg/kg, 8.0 mg/kg, 7.5 mg/kg, 7.0 mg/kg, 6.5 mg/kg, 6.0 mg/kg, 5.5 mg/kg, 5.0 mg/kg, 4.5 mg/kg, 4.0 mg/kg, 3.5 mg/kg, 3.0 mg/kg, 2.5 mg/kg, or 2.0 mg/kg. In certain embodiments, an Mcl-1 inhibitor can be administered at a dosage range of 10 mg/kg to 8 mg/kg, 9.5 mg/kg to 7.5 mg/kg, 9.0 mg/kg to 7.0 mg/kg, 8.5 mg/kg to 6.5 mg/kg, 8.0 mg/kg to 6.0 mg/kg, 7.5 mg/kg to 5.5 mg/kg, 7.0 mg/kg to 5.0 mg/kg, 6.5 mg/kg to 4.5 mg/kg, 6.0 mg/kg to 4.0 mg/kg, 5.5 mg/kg to 3.5 mg/kg, 5.0 mg/kg to 3.0 mg/kg, 4.5 mg/kg to 2.5 mg/kg, or 4.0 mg/kg to 2.0 mg/kg. In a specific embodiment, an Mcl-1 inhibitor is administered at a dosage amount of 5.0 mg/kg. In a specific embodiment, an Mcl-1 inhibitor is administered at a dosage amount of 7.5 mg/kg. In certain embodiments, the same dosage amount is administered each day. In certain embodiments, the dosage amount administered is not the same.

In certain embodiments, examples of an Mcl-1 inhibitor can be, but are not limited to, obatoclax, A-1210477, AMG176, S64315, S63845, or AZD5991. In a specific embodiment, an Mcl-1 inhibitor is S63845. In certain embodiments, an inhibitor of Bcl-2 is venetoclax (ABT-199), oblimersen, PNT2258, or SPC2996. In a specific embodiment, an inhibitor of Bcl-2 is venetoclax (ABT-199). In certain embodiments, the patients can also treated with ATG before the transplant and anti-CD154 and cyclosporine post-transplant. In a specific embodiment, a Bcl-2 inhibitor and an Mcl-1 inhibitor are administered such to achieve sustained chimerism in a recipient.

In certain embodiments, induced chimerism can last for a period of time, including at least 1 month, 2 months, 3 months, 4 months, 5 months, or at least 6 months. In a specific embodiment, the chimerism will persist in the recipient for at least 6 months.

### 6.4.6 Conditioning Regimen Comprising an Mcl-1 Inhibitor

In a certain embodiment, an inhibitor of Mcl-1 may be administered in a treatment regimen without an additional Bcl-2 inhibitor. In certain embodiments, an Mcl-1 inhibitor may be administered to the patient beginning 10 days before transplant, beginning 9 days before transplant, beginning 8 days before transplant, beginning 7 days before transplant, beginning 6 days before transplant, beginning 5 days before transplant, beginning 4 days before transplant, beginning 3 days before transplant, beginning 2 days before transplant, beginning 1 day before transplant, or beginning the day of transplant. In certain embodiments, an Mcl-1 inhibitor is administered daily. In certain embodiments, an Mcl-1 inhibitor is administered more than once per day. In a specific embodiment, administration of an Mcl-1 inhibitor may be administered to the patient beginning 6 days before transplant. In certain embodiments, an inhibitor of Mcl-1 may be administered for a time course of 10 days, 9 days, 8 days, 7 days, 6 days, 5 days, 4 days, 3 days, 2 days, or 1 day. In a specific embodiment, an inhibitor of Mcl-1 can be administered for 5 days. In a specific embodiment, an inhibitor of Mcl-1 can be administered to a patient for a time course of 5 days beginning 6 days before the transplant.

In certain embodiments, an Mcl-1 inhibitor can administered at a dosage of 10 mg/kg, 9.5 mg/kg, 9.0 mg/kg, 8.5 mg/kg, 8.0 mg/kg, 7.5 mg/kg, 7.0 mg/kg, 6.5 mg/kg, 6.0 mg/kg, 5.5 mg/kg, 5.0 mg/kg, 4.5 mg/kg, 4.0 mg/kg, 3.5 mg/kg, 3.0 mg/kg, 2.5 mg/kg, or 2.0 mg/kg. In certain embodiments, an Mcl-1 inhibitor can be administered at a dosage range of 10 mg/kg to 8 mg/kg, 9.5 mg/kg to 7.5 mg/kg, 9.0 mg/kg to 7.0 mg/kg, 8.5 mg/kg to 6.5 mg/kg, 8.0 mg/kg to 6.0 mg/kg, 7.5 mg/kg to 5.5 mg/kg, 7.0 mg/kg to 5.0 mg/kg, 6.5 mg/kg to 4.5 mg/kg, 6.0 mg/kg to 4.0 mg/kg, 5.5 mg/kg to 3.5 mg/kg, 5.0 mg/kg to 3.0 mg/kg, 4.5 mg/kg to 2.5 mg/kg, or 4.0 mg/kg to 2.0 mg/kg. In a specific embodiment, an Mcl-1 inhibitor is administered at a dosage amount of 5.0 mg/kg. In a specific embodiment, an Mcl-1 inhibitor is administered at a dosage amount of 7.5 mg/kg. In certain embodiments, the same dosage amount is administered each day. In certain embodiments, the dosage amount administered is not the same. In certain embodiments, examples of an Mcl-1 inhibitor can be, but are not limited to, obatoclax, A-1210477, AMG176, S64315, S63845, or AZD5991. In a specific embodiment, an Mcl-1 inhibitor is S63845. In certain embodiments, the patients can also treated with ATG before the transplant and anti-CD154 and cyclosporine post-transplant. In a specific embodiment, an Mcl-1 inhibitor is administered such to achieve sustained chimerism in a recipient.

In certain embodiments, induced chimerism can last for a period of time, including at least 1 month, 2 months, 3 months, 4 months, 5 months, or at least 6 months. In a specific embodiment, the chimerism will persist in the recipient for at least 6 months.

### 6.5 Transplantation

The procedure for obtaining and implanting the organ is well-known to the skilled artisan. Any procedure for the surgical removal from the donor and the surgical implantation in the recipient can be used with the methods provided herein. In certain embodiments, the organ can be treated between removal and implantation.

### 6.6 Bone Marrow or HSC Infusion

The procedures for obtaining and infusing bone marrow or HSC is well-known to the skilled artisan. In certain embodiments, the donor bone marrow is unprocessed. In certain embodiments, at least 1×, 2×, 3×, 4×, 5×, 6×, 7×, 8×, or at least 9×10⁷ cells/kg are infused. In certain embodiments, at least 1×, 2×, 3×, 4×, 5×, 6×, 7×, 8×, or at least 9×10⁸ cells/kg are infused.

In some embodiments, the donor of the organ and the donor of the bone marrow (HSCs) are the same person or the donor of the bone marrow (HSCs) is a person that is HLA-identical to the organ donor. In some embodiments, the organ is HLA mismatched.

In some embodiments, the bone marrow (HSCs) and organ transplantation can be performed on the same day. In some embodiments, the bone marrow and organ transplantation can be performed on the concurrently. In some embodiments, the bone marrow transplantation can be performed subsequent to the organ transplantation or vice versa.

### 6.7 Postoperative Treatment Regimen

Postoperative treatment regimens for use with the methods provided herein can include administration of one or more of the treatments below.

### 6.7.1 Cyclosporine

Cyclosporine A (CyA), as used herein, is a compound administered to a recipient to suppress the immune system, with a specific action on T cells. CyA (C₆₂H₁₁₁N₁₁O₁₂) can be administered to a transplant recipient to inhibit the development of Graft versus Host disease. Brand names of CyA include Gengraf®, Neoral®, and Sandimmune®.

In certain embodiments, the conditioning regimen provided herein comprises administering CyA to a transplant recipient. In certain embodiments, CyA can be administered 1 day before the transplant, 2 days before the transplant, 3 days before the transplant, 1 day and 2 days before the transplant, 1 day and 3 days before transplant, or 1 day and 2 days and 3 days before the transplant. In a specific embodiment, CyA can be administered to a recipient 1 day before the transplant.

In certain embodiments, a single dose amount of CyA can be administered. In certain embodiments, multiple dose amounts of the CyA can be administered. In certain embodiments, a constant dose of CyA can be administered. In certain embodiments, a tapering course of CyA can be administered. In certain embodiments, a constant dose of CyA followed by a tapering course of CyA can be administered.

In certain embodiments, CyA can be administered to a transplant recipient at a dose amount of 2 mg/kg/day, 2.5 mg/kg/day, 3 mg/kg/day, 3.5 mg/kg/day, 4 mg/kg/day, 4.5 mg/kg/day, 5 mg/kg/day, 5.5 mg/kg/day, 6 mg/kg/day, 6.5 mg/kg/day, 7 mg/kg/day, 7.5 mg/kg/day, 8 mg/kg/day, 8.5 mg/kg/day, 9 mg/kg/day, 9.5 mg/kg/day, 10 mg/kg/day, 10.5 mg/kg/day, 11 mg/kg/day, 11.5 mg/kg/day, 12 mg/kg/day, 12.5 mg/kg/day, 13 mg/kg/day, 13.5 mg/kg/day, 14 mg/kg/day, 14.5 mg/kg/day, 15 mg/kg/day, 15.5 mg/kg/day, 16 mg/kg/day, 16.5 mg/kg/day, 17 mg/kg/day, 17.5 mg/kg/day, 18 mg/kg/day, 2-6 mg/kg/day, 3-7 mg/kg/day, 4-8 mg/kg/day, 5-9 mg/kg/day, 6-10 mg/kg/day, 7-11 mg/kg/day, 8-12 mg/kg/day, 9-13 mg/kg/day, 10-14 mg/kg/day, 11-15 mg/kg/day, 12-16 mg/kg/day, 13-17 mg/kg/day, or 14-18 mg/kg/day. In a specific embodiment, CyA can be administered to a transplant recipient at a dose amount of 8 mg/kg/day. In a specific embodiment, CyA can be administered to a transplant recipient at a dose amount of 9 mg/kg/day. In a specific embodiment, CyA can be administered to a transplant recipient at a dose amount of 10 mg/kg/day. In a specific embodiment, CyA can be administered to a transplant recipient at a dose amount of 11 mg/kg/day. In a specific embodiment, CyA can be administered to a transplant recipient at a dose amount of 12 mg/kg/day. In a specific embodiment, CyA can be administered to a transplant recipient at a dose range of 8-12 mg/kg/day.

In certain embodiments, CyA can be administered to a recipient at a sufficient dose amount to obtain the target trough blood levels of 100-200 ng/ml, 125-225 ng/ml, 150-250 ng/ml, 175-275 ng/ml, 200-300 ng/ml, 225-325 ng/ml, 250-350 ng/ml, 275-375 ng/ml, 300-400 ng/ml, 325-425 ng/ml, 350-450 ng/ml, 375-475 ng/ml, or 400-500 ng/ml. In a specific embodiment, the target trough blood levels can be 250-350 ng/ml.

In certain embodiments, CyA can be administered to a transplant recipient in a convenient manner known in the art including subcutaneously, intravenously, intravascularly, topically, intraarterially, intracranially, intramuscularly, orally, intraorbitally, by inhalation, transdermally, or intraperitonially, or through a route of administration which allows for the proper action of the CyA by the recipient. In a specific embodiment, CyA can be administered orally.

In certain embodiments, substitute compounds can be used in the place of CyA. These compounds can include tacrolimus (Prograf®, Adport®, Advagraf®, Envarsus®, Modigraf®, Astagraf®), Belatacept (Nulojix®), sirolumus, and everolimus. In specific embodiments, Belatacept can be administered to suppress the immune system of the recipient.

### 6.8 Anti-Apoptotic Bcl-2 Inhibitors

In certain embodiments, the post-operative treatment can include administering an inhibitor of an anti-apoptotic Bcl-2 family member. In certain, more specific embodiments, the administration of such an inhibitor is a second course of treatment with such an inhibitor in the transplantation regimen (i.e., the second course following a treatment “holiday” after administration of such an inhibitor during the conditioning of the transplant recipient). Such inhibitors and their dosing regimens are described in Section 6.4.1.

### 6.9 Pharmaceutical Compositions

The present invention includes inhibitors of an anti-apoptotic Bcl-2 family member (e.g., Bcl-2 or Mcl-1) described herein, and/or additional agents in various formulations of pharmaceutical composition. Any inhibitor of an anti-apoptotic Bcl-2 family member (e.g., Bcl-2 or Mcl-1), described herein, can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In embodiments, the composition is in the form of a capsule or a tablet. Other examples of suitable pharmaceutical excipients are described in Remington's Pharmaceutical Sciences 1447-1676 (Alfonso R. Gennaro eds., 19th ed. 1995), incorporated herein by reference.

Pharmaceutical compositions comprising an inhibitor of an anti-apoptotic Bcl-2 family member (e.g., Bcl-2 or Mcl-1), described herein, and/or additional agents can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The pharmaceutical compositions comprising an inhibitor of an anti-apoptotic Bcl-2 family member (e.g., Bcl-2 or Mcl-1) described herein, and/or additional agents of the present invention may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any inhibitor of an anti-apoptotic Bcl-2 family member (e.g., Bcl-2 or Mcl-1) described herein, and/or additional agents disclosed herein is formulated in accordance with the methods described herein.

### 6.10 Clinical Outcome Assessment

In certain embodiments, the methods described herein can be assayed and analyzed by known methods in the art.

In certain embodiments, methods described herein are beneficial for an improved clinical outcome and response of the patient or recipient. In certain embodiments, a clinical outcome of the methods provided herein results in hematopoieticchimerism without neutropenia or with reduced neutropenia. In certain embodiments, the methods provided herein results in hematopoietic chimerism with a reduced incidence of GVHD in the recipient. In certain embodiments, the methods provided herein results in hematopoietic chimerism with a reduced risk of severity of GVHD in the recipient. In certain embodiments, the methods provided herein results in no transplant rejection in the patient. In certain embodiments, the methods provided herein results in the prevention of myelosuppression in the patient. In certain embodiments, the methods provided herein results in induced hematopoietic chimerism in the patient which can persist indefinitely after the bone marrow transplantation. In certain embodiments, this method can be used for bone marrow or HSC transplant with gene transfer.

### 6.10.1 Neutropenia

In certain embodiments, hematopoietic chimerism without neutropenia or with reduced neutropenia as described herein can be assayed and analyzed by known methods in the art, for example, colony forming cell (CFC) assays. Neutropenia may be diagnosed by a blood cell count performed on a sample of blood removed from, for example, a vein. Bone marrow biopsy may be used to diagnose the specific cause of neutropenia. In certain embodiments, the methods provided herein results in hematopoietic chimerism without neutropenia or with reduced neutropenia. In certain embodiments, a clinical response of the methods provided herein results in lymphocyte depletion but without neutropenia. In certain embodiments, the methods described herein results in at least 5%, 10%, 15%, 20%, 25%, 30%, 35%, 40%, 45%, 50%, 55%, 60%, 65%, 70%, 75%, 80%, 85%, 90%, 95%, or even 100% less neutropenia as compared to the same treatment regimen in the absence of administration of an inhibitor of an anti-apoptotic member of the Bcl-2 protein family.

6.10.2 Reduced Graft vs. Host Disease

In some embodiments, the incidence of GVHD in the recipient is reduced in the clinical response of the methods described herein. In some embodiments, the severity and/or degree of GVHD is reduced in the recipient. In some embodiments, the incidence of GVHD is reduced by about 5%, 10%, 15%, 20%, 25%, 30%, 35%, 40%, 45%, 50%, 55%, 60%, 65%, 70%, 75%, or 80%, 85%, 95% as compared to no treatment regimen in the recipient. In some embodiments, the clinical outcome results in allograft tolerance. In some embodiments, the severity and/or degree of GVHD in the recipient by is reduced by about 5%, 10%, 15%, 20%, 25%, 30%, 35%, 40%, 45%, 50%, 55%, 60%, 65%, 70%, 75%, or 80%, 85%, 95% as compared to no treatment regimen in the recipient.

In certain embodiments, the clinical outcome results in induced hematopoietic chimerism in the patient which can persist indefinitely after the bone marrow transplantation.

### 6.10.3 Reduced Immunosuppression

In certain embodiments, the desired outcome allows for the reduction of the amount of TBI necessary to achieve immune tolerance towards the organ. In certain embodiments, the dosage of TBI administered to a recipient is at most 1.5 Gy, 1.4 Gy, 1.3 Gy, 1.2 Gy, 1.1 Gy, 1.0 Gy, 0.9 Gy, 0.8 Gy, 0.7 Gy, 0.6 Gy, 0.5 Gy, 0.4 Gy, 0.3 Gy, 0.2 Gy, or 0.1 Gy. In certain embodiments, the desired outcome allows for the elimination of the TBI requirement in order to achieve immune tolerance towards the organ.

In certain embodiments, a clinical outcome of the methods provided herein results in the prevention of myelosuppression. In certain embodiments, prevention of myelosuppression as described herein can be assayed and analyzed by known methods in the art. For example, functional in vitro myelosuppression and hematotoxicity assays such as colony forming cell (CFC) assays using normal human bone marrow grown in appropriate semi-solid media such as Colony GEL have been shown to be useful in examining clinical myelotoxicity.

### 6.11 Kits

Aspects of the present invention provide kits that can simplify the administration of the pharmaceutical compositions and/or chimeric proteins disclosed herein.

An illustrative kit of the invention comprises any inhibitor of an anti-apoptotic Bcl-2 family member (e.g., Bcl-2 or Mcl-1), described herein, and/or pharmaceutical composition disclosed herein in unit dosage form. In embodiments, the unit dosage form is a container, such as a pre-filled syringe, which can be sterile, containing any agent disclosed herein and a pharmaceutically acceptable carrier, diluent, excipient, or vehicle. The kit can further comprise a label or printed instructions instructing the use of any agent disclosed herein. The kit may also include a lid speculum, topical anesthetic, and a cleaning agent for the administration location. The kit can also further comprise one or more additional agent disclosed herein. In embodiments, the kit comprises a container containing an effective amount of a composition of the invention and an effective amount of another composition, such those disclosed herein. Aspects of the present invention include use of an inhibitor of an anti-apoptotic Bcl-2 family member (e.g., Bcl-2 or Mcl-1) as disclosed herein in the manufacture of a medicament, e.g., a medicament for inducing hematopoietic chimerism in a patient in need thereof.

### 6.11.1 Kits and Systems

Any aspect or embodiment disclosed herein can be combined with any other aspect or embodiment as disclosed herein.

In certain embodiments, a kit and system described herein may comprise a specific inhibitor of Bcl-2. In certain embodiments, the kit and systems described herein may comprise a specific inhibitor of Mcl-1. In certain embodiments, the kit and systems described herein may comprise any combination of a specific inhibitor of Bcl-2 and a specific inhibitor of Mcl-1. For example, a kit and system described herein may comprise a specific inhibitor of Bcl-2 and a specific inhibitor of Mcl-1.

In certain embodiments, a kit and system described herein may comprise a blister pack of a specific inhibitor of an anti-apoptotic Bcl-2 family member with the number of tablets needed for one transplantation regiment. In certain embodiments, a kit and system described herein may comprise a blister pack of a specific inhibitor of Bcl-2 with the number of tablets needed for one transplantation regiment. In certain embodiments, a kit and system described herein may comprise a blister pack of a specific inhibitor of Mcl-1 with the number of tablets needed for one transplantation regiment.

In some embodiments, a kit and system described herein may comprise an inhibitor of an anti-apoptotic Bcl-2 family member and ATG. In certain embodiments, the kit and system described herein may comprise a specific inhibitor of Bcl-2 and ATG. In certain embodiments, the kit and system described herein may comprise a specific inhibitor of Mcl-1 and ATG. In some embodiments, a kit and system described herein may comprise an inhibitor of an anti-apoptotic Bcl-2 family member and CyA. In certain embodiments, the kit and system described herein may comprise a specific inhibitor of Bcl-2 and CyA. In certain embodiments, the kit and system described herein may comprise a specific inhibitor of Mcl-1 and CyA.

### 6.12 Equivalents and Incorporation by Reference

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

### 7. EXAMPLES

The invention is further described in the following examples, which do not limit the scope of the invention described in the claims.

### Example 1: Methods for Inducing Hematopoietic Chimerism in a Patient

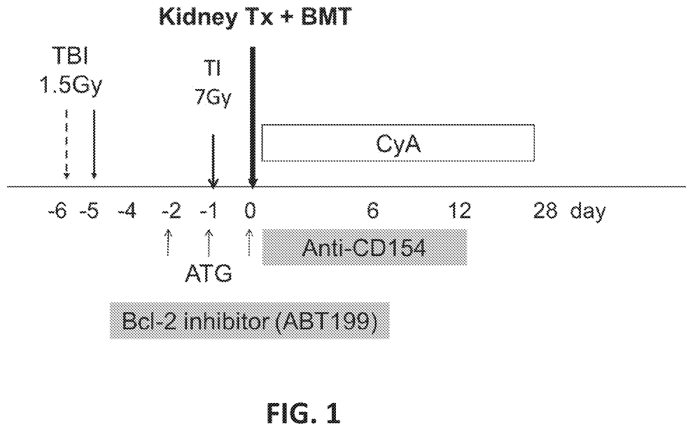

Prior to bone marrow and kidney transplantation, the patient needing treatment is administered total body irradiation (TBI) at 0.1 to 1.5 Gy shortly before transplant (e.g. on days 5 and 4). On days 2, and 1, a specific anti-apoptotic Bcl-2 inhibitor (e.g., venetoclax and/or S64315) is administered to the patient on a dosing regimen of about 11 unit doses at 10 mg/kg. Thymic radiation is administered at 7 Gy on days 2 and 1 before bone marrow and kidney transplantation. A post-operative treatment with Cyclosporine A (CyA), may be administered to the patient. See FIG. 1.

### Example 2: Bcl-2 Inhibition with Venetoclax Promotes Induction of Hematopoietic Chimerism and Renal Allograft Tolerance without Severe Myelosuppression in Non-Human Primate

Induction of specific immunologic tolerance is an ultimate goal of organ transplantation. Induction of hematopoietic chimerism achieved via donor bone marrow transplantation (DBMT) has so far been the only method able to reproducibly induce tolerance in clinical transplantation. In previous studies, total body irradiation (TBI) dose less than 3.0 Gy has never consistently achieved engraftment of hematopoietic stem cells with nonmyeloablative conditioning regimen even with various other combined treatments. However, administration of TBI 3.0 Gy resulted in non-specific deletion of the entire hematopoietic cells, which may be associated with infectious complications or post-transplant lymphoproliferative disease (PTLD) after DBMT.

Cippà et al., Pharmacological modulation of cell death in organ transplantation. Transplant international, 2017, recently found that persistent hematopoietic chimerism and skin allograft tolerance across a full MHC disparity can be induced without any immunosuppressive treatment in mice by using B cell lymphoma-2 (Bcl-2) inhibitor in combination with costimulatory blockade (CoB) and cyclosporine (CyA). In the original mouse study, CoB with anti-CD154 mAb appeared to be essential to induce persistent hematopoietic chimerism by the conditioning regimen with Bcl-2 inhibition.

In a mice BMT model with CoB, massive peripheral deletion of donor reactive T cells was shown to occur in the early period (1 week) after BMT. This deletion was inhibited in Fas-deficient and in Bcl-xL transgenic recipients, which indicates involvement of both extrinsic (AICD) and intrinsic (Bcl-2) apoptosis for early peripheral deletion of donor reactive T cells. The major objective of this study is to develop a nontoxic conditioning regimen with BCl-2 inhibition to induce robust allograft tolerance via the hematopoietic chimerism approach without myelosuppression.

Methods

To evaluate whether Bcl-2 inhibitor (ABT-199) can promote engraftment of hematopoietic stem cells with reduced myelosuppressive therapy, monkeys were treated with various modified regimens with ABT-199 and compared with standard regimen without ABT-199. The basic conditioning regimen consisted of low dose total body irradiation (TBI), thymic irradiation (TI, 7 Gy) and peri-transplant ATG administration. After combined kidney and bone marrow transplantation, the recipients were treated with anti-CD154 mAb for 2 weeks and CyA for 4 weeks, after which no immunosuppression was given (FIG. 1). In Groups C-E, 10 mg/kg of ABT-199 was administered for 11 days (day −4 to day 6). Animals were divided into five groups according to the treatments (Table 1).

To examine intrinsic apoptosis induced by ABT-199, expression of BAX (the BCL-2-associated X protein), which is pro-apoptotic protein that plays an important role in the mitochondria-dependent apoptotic pathway was assessed.

Results

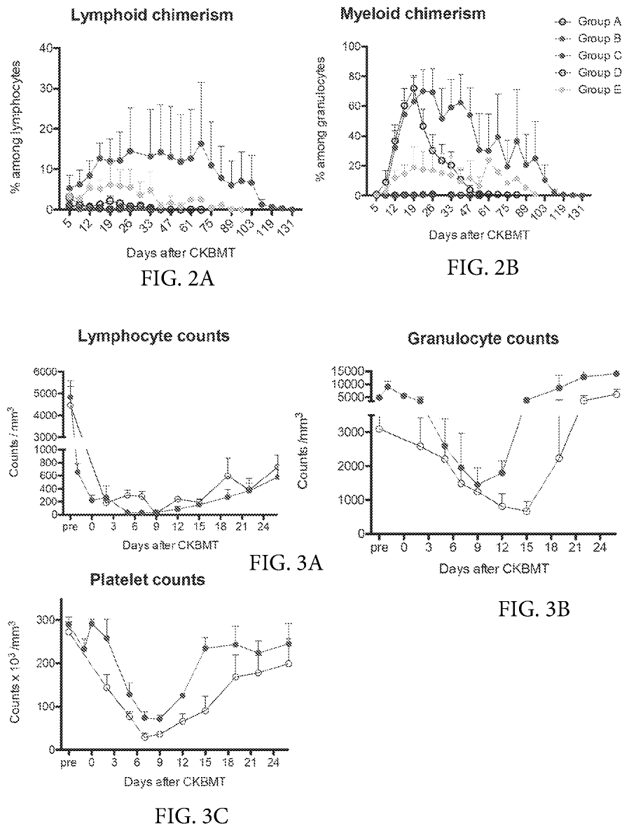

With 3 Gy TBI (Group A), successful induction of myeloid dominant transient hematopoietic chimerism (FIGS. 2A-B) and renal allograft tolerance was achieved (Table 2). However, recipients developed transient but severe pancytopenia (FIG. 3A-C). In Group B, the TBI dose was reduced to 1.5 Gy. All three recipients in Group B failed to develop chimerism. By adding ABT-199 to Group B, 3/3 recipients in Group C developed significantly higher and longer hematopoietic chimerism, especially in the lymphoid lineage (FIG. 2), without development of severe pancytopenia (FIG. 3). All recipients in Group C achieved long-term immunosuppression free renal allograft survival (Table 2). In Group D, both recipients failed to develop chimerism and rejected their allografts on day 142 and 100 due to acute cellular rejection. Although three Group E recipients successfully developed chimerism, all rejected their renal allografts on day 100, 97, 163 with early recovery of CD31⁺ naïve T cells, see Table 2.

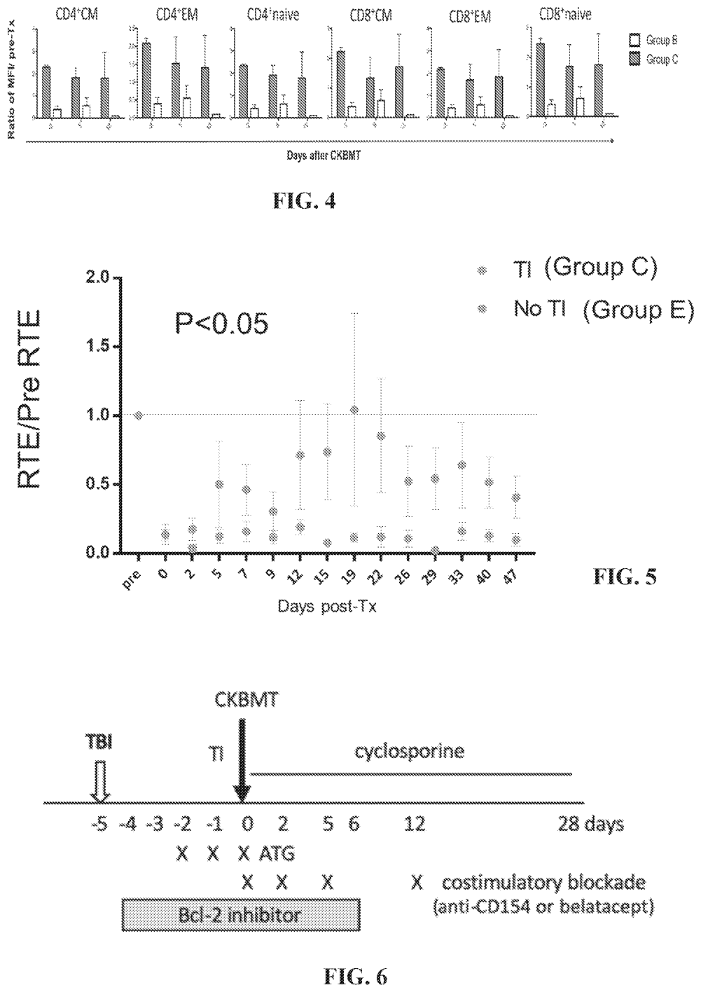

Bcl-2 inhibition effectively induced apoptosis of T cell subset: Expression of BAX in Group C was consistently higher than those in Group B after treatment of ABT-199, indicating that higher levels of intrinsic apoptosis were induced in CD4 and CD8 T cells by adding ABT-199, see FIG. 4.

Overall, enhancement of intrinsic apoptosis with Bcl-2 inhibition is a promising strategy to achieve robust hematopoietic chimerism and allograft tolerance without causing myelosuppressive complications.

While the animals of Group E developed hematopoietic chimerism, all rejected their allografts (day 100, 97, and 163) due to chronic rejection. The difference in treatment protocol between Group E and Group C (which also developed hematopoietic chimerism and achieved tolerance) was that the animals of Group C received a dose of thymic irradiation. As shown in FIG. 5, the dose of thymic irradiation given to Group C was sufficient to completely suppress the recovery of recent thymice emigrants (RTEs).

RTEs (CD4⁺CD45RA⁺CD31⁺) represent naive T-cells that have recently migrated from the thymus to the lymphoid periphery. The RTEs will become mature naïve T cells progressively over the course of 2-3 weeks. In the experiment described above, RIEs were the only subset of T cells that differed between the animals of Group C and those of Group E. The RTEs were completely suppressed in recipients of thymic irradiation (Group C) for as long as 50 days, while RTEs returned to the pre transplant levels earlier and were significantly higher in the animals that did not receive thymic irradiation (Group E), as measured by the ratio of post-transplant RTEs relative to pre-transplant levels.

The presence of these RTEs, while they do not inhibit induction of hematopoietic chimerism, may have prevented tolerance induction in Group E and thus contribute to allograft rejection. Thymic irradiation appeared to be necessary for induction of renal allograft tolerance despite successful induction of hematopoietic chimerism.

TABLE 1
Experimental Groups

| Group | N | ABT-199 | TBI (Gy) | TI (Gy) |
|---|---|---|---|---|
| A | 8 | − | 3 | 7 |
| B | 3 | − | 1.5 | 7 |
| C | 3 | + | 1.5 | 7 |
| D | 2 | + | 0 | 7 or 0 |
| E | 3 | + | 1.5 | 0 |

TABLE 2
The results of ABT-199 with various condition regimen and standard regimen

| | ABT- | TBI | TI | | Graft survival | |
|---|---|---|---|---|---|---|
| Group | 199 | (Gy) | (Gy) | Chimerism | (Days) | Outcome |
| A | − | 3 | 7 | 7/8 | 2498, 4328, 837, | 4: no rejection |
| | | | | | 755, 401, 373, | 3: chronic rejection |
| | | | | | 206, 58 | 1: acute rejection |
| B | − | 1.5 | 7 | 0/3 | >58, >81, >109 | in progress |
| C | + | 1.5 | 7 | 3/3 | >313, >361, >646 | no rejection |
| D | + | 0 | 7 | 0/2 | 142, 100 | 2: acute rejection |
| E | + | 1.5 | 0 | 2/2 | 100, 97, 163 | 3: chronic rejection |

### Example 3: Bcl-2 Inhibition with Venetoclax Promotes Hematopoietic Chimerism and Allograft Tolerance without Myelosuppression

Despite marked improvement in short-term results in organ transplantation, life-long administration of potent immunosuppressive drugs limits the life expectancy and quality of life of recipients by increasing the risk of cardiovascular disease (Morales, J. M. & Dominguez-Gil, B. 2005, J Hypertens. 23, 1609-1616.; Miller, L. W. 2002, Am J Transplant 2, 807-818.), infectious complications (Fishman, J. A. & Rubin, R. H. 1998, N Engl J Med 338, 1741-1751.; Alangaden, G. J., et al. 2006, Clin Transplant 20, 401-409.), malignancies (Kasiske, B. L., et al. 2004, Am J Transplant 4, 905-913.) and various metabolic derangements (Jindal, R. M., Sidner, R. A. & Milgrom, M. L. 1997, Drug Saf 16, 242-257.; Marchetti, P. & Navalesi, R. 2000, J Endocrinol Invest 23, 482-490.). Moreover, chronic rejection is not consistently prevented even with potent immunosuppression (Pascual, M., et al. 2002, N Engl J Med 346, 580-590.). Consequently, the 10-year renal allograft survival rate after deceased donor kidney transplantation (KTx) remains around 50%, with as many as 25% of recipients dying despite ongoing graft function (Briganti, E. M., et al. 2002, N Engl J Med 347, 103-109.; OPTN/SRTR. 2016, Am J Transpl 16, 4-215.). Therefore, induction of allograft tolerance remains an ultimate goal of organ transplantation to overcome the limitations of current therapy with chronic immunosuppression. Among the tolerance induction protocols tested to date, induction of hematopoietic chimerism through donor bone marrow transplantation (DBMT) is the only approach that has provided repeatedly successful immunosuppression-free allograft survival for human renal allograft recipients (Spitzer, T. R., et al. 1999, Transplantation 68, 480-484.; Fudaba, Y., et al. 2006, Am J Transplant 6, 2121-2133.; Kawai, T., et al. 2008, N Engl J Med 358, 353-361.; Millan, M. T., et al. 2002, Transplantation 73, 1386-1391.; Leventhal, J., et al. 2012, Sci Transl Med 4, 124ra128.; Kawai, T., et al. 2019, Am J Transplant 19, 324-330.; Scandling, J. D., et al. 2015, Am J Transplant 15, 695-704.). However, more widespread clinical application of a tolerance induction approach via hematopoietic chimerism has been hampered by the myelosuppressive toxicity of the current conditioning therapies (e.g. total body irradiation or cyclophosphamide). The resultant pancytopenia required intensive antimicrobial prophylaxis, isolation precautions, and extended hospitalization. For wider clinical application of tolerance protocols, it is imperative to develop a novel approach to induce chimerism without causing severe myelosuppression.

Materials and Methods

Animals and pair selections: Cynomolgus monkeys, including donor animals, weighing 4-8 kg were used for this study (Charles River Primates, Wilmington, Mass.). Donors and recipients were paired on the basis of ABO blood type compatibility and major histocompatibility complex (MHC) mismatching. MHC characterization was performed as previously described (O'Connor, S. L., et al. 2007, Immunogenetics 59, 449-462.; Pendley, C. J., et al. 2008, Immunogenetics 60, 339-351.). All surgical procedures and postoperative care of animals were performed in accordance with National Institute of Health guidelines for the care and use of primates and were approved by the Massachusetts General Hospital Institutional Animal Care and Use Committee (IACUC).

Combined kidney and bone marrow transplantation (CKBMT): All recipients were conditioned with total body irradiation (TBI) (1.5 Gy on day −5, relative to the day of CKBMT), local thymic irradiation (TI) (7 Gy on day −1) and equine-derived anti-thymocyte globulin (hATG) (Atgam; Pharmacia and Upjohn, Kalamazoo, Mich.; 50 mg/kg/day i.v. on days −2, −1 and 0). Following DBMT, the recipients were also treated with anti-CD154 mAb (5c8; 20 mg/kg i.v. on days 0 and 2, 7, and 12 post-DBMT). Cyclosporine A (CyA, Novartis, Basel, Switzerland) was administered intramuscular (IM) on days 0-27 post DBMT, after which no immunosuppression was administered (FIG. 6). A dose of 10 mg/kg of a Bcl-2 inhibitor, either venetoclax (ABT-199, Selleckchem, Houston, Tex.) or navitoclax (ABT-263, Selleckchem, Houston, Tex.), was administered intramuscularly daily from day −4 to day +6.

Bone marrow transplantation: Donor bone marrow cells (DBMC) were obtained by multiple aspirations from the iliac crests, humerus head, and vertebral bones under general anesthesia. If the donor animal was sacrificed, DBMC were harvested from the vertebral bones after euthanasia. DBMC (1.0-3.0×10⁸ mononuclear cells/kg) were infused intravenously.

Kidney transplantation (KTx): Monkeys underwent heterotopic KTx and nephrectomy as previously described (Cosimi, A. B., et al. 1990, Surgery 108, 406-413.).

Flow cytometric and CyTOF analyses: Peripheral blood mononuclear cells (PBMCs) were labeled with a combination of the following mAbs: CD3 (SP34-2), CD4 (L200), CD8 (SK1), CD21 (B-ly4), CD27 (M-T271), CD28 (CD28.2), CD95 (DX2), and IgM (G20-127) (BD Pharmingen, San Jose, Calif.), CD20 (2H7) (Biolegend, Inc., San Diego, Calif.) and FOXP3 (236A/E7) (eBioscience, Inc., San Diego, Calif.). The fluorescence of the stained samples was analyzed using FACSverse (BD Biosciences, San Jose, Calif.) and Accuri flow cytometers (BD PharMingen), and FlowJo software (Tree Star, Inc., Ashland, Oreg.). A CyTOF panel for cynomolgus monkeys was designed. The CyTOF panel consisted of antibodies against various NHP leukocyte markers including CD3, CD4, CD8, Foxp3, CD127, CTLA4, Ki-67, CD95, CD20, CD21, CD27, CD38, CD123, CD31, CD11c, CD25, HLA-DR, CD45RA, CD159 (NKG2a), Granzyme B, Caspase 3/7/9, Bax and Cytochrom C.

Detection of hematopoietic chimerism: A monkey was selected that was either BB7.6 (HLA-B7B40) positive or a H38 (HLA-BW6) positive monkey as a donor and a monkey negative to these antibodies was selected as a recipient. In all experiments, the percentage of cells that stained with each mAb was determined from one color fluorescence histogram and compared with those obtained from donor and pretreatment frozen recipient cells, which were used as positive and negative controls. The percentage of cells considered positive was determined with a cutoff chosen as the fluorescence level at the beginning of the positive peak for the positive control stain and by subtracting the percentage of cells stained with an isotype control. By using forward and 90° light scatter (FSC and SSC, respectively) dot plots, lymphocyte (FSC- and SSC-low), granulocyte (SSC-high), and monocyte (FSC-high but SSC-low) populations were gated, and chimerism was determined separately for each population. Nonviable cells were excluded by propidium iodide (Thermo Fischer Scientific, Grand Island, N.Y.) staining.

Statistical analyses: Statistical analysis was performed with GraphPad PRISM 7.01 (GraphPad Software, Inc, San Diego, Calif.). We used two way ANOVA to compare chimerism, CBC, and T cell subsets. Mantel-Cox log-rank test was used to analyze survival of different groups and. P values lower than 0.05 have been considered statistically significant.

Results

Selective Bcl-2 Inhibition with Venetoclax Induced Superior Apoptosis of Lymphocytes Compared to Navitoclax that Inhibits Bcl-2, Bcl-xL and Bcl-w

Two clinically tested Bcl-2 inhibitors, ABT-263 (navitoclax) and ABT-199 (venetoclax), were evaluated for their ability to enhance apoptosis of lymphocytes. Navitoclax has multiple affinities, to Bcl-2, Bcl-xl and Bcl-w, similar to ABT-737 that was used in the murine study, but has superior efficacy and bioavailability to ABT-737 (Gandhi, L., et al. 2011, J Clin Oncol 29, 909-916.). Venetoclax is approved by FDA for treatment of hematologic malignancies, and is highly selective to Bcl-2 (Souers, A. J., et al. 2013, Nat Med 19, 202-208.).

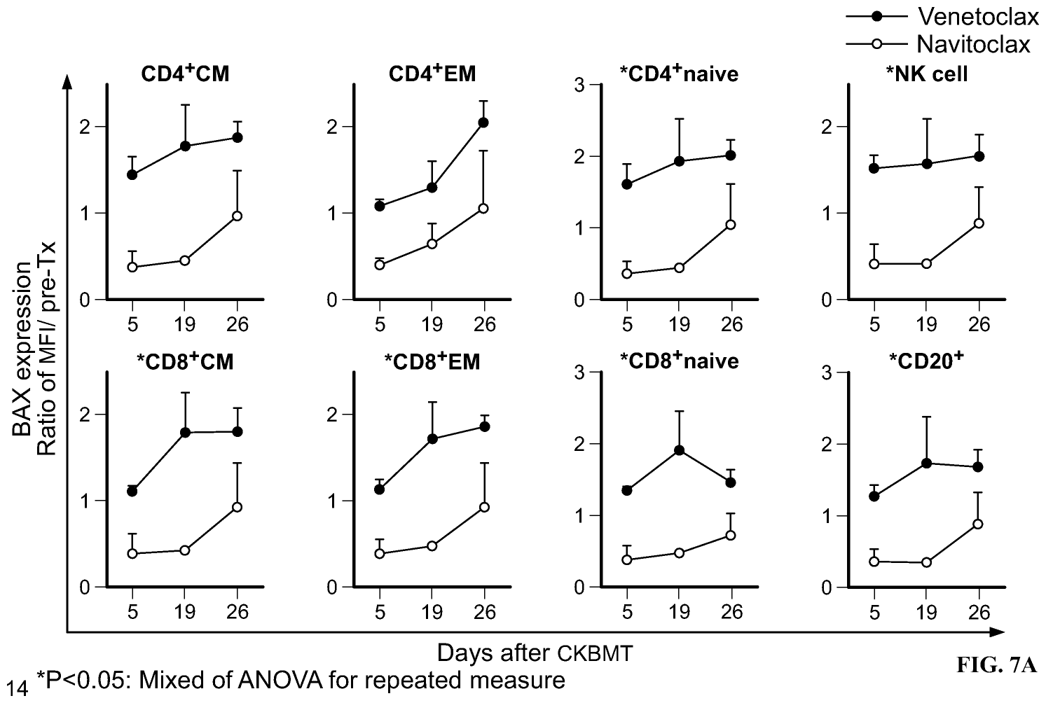

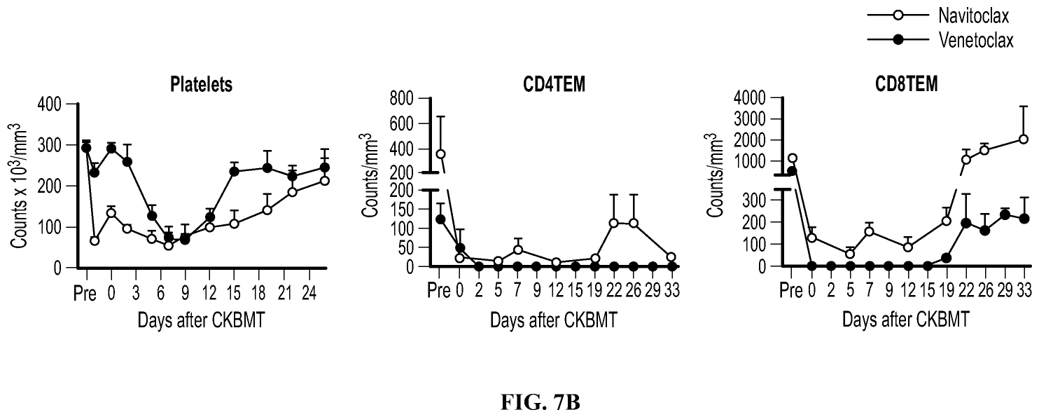

To evaluate the Bcl-2 inhibitors, the inhibitors were incorporated into a nonmyeloablative conditioning regimen that has been shown to induce chimerism and renal allograft tolerance^{23,24} but with reduced total body irradiation (TBI) dose. This regimen consisted of low dose TBI, local thymic irradiation (TI), anti-thymocyte globulin (ATG), post-transplant administration of costimulatory blockade, and a one month course of cyclosporine. Peri-transplant administration (10 mg/kg administered daily from four days before transplant (day −4) through six days after transplant (day +6) of either navitoclax or venetoclax was added to the conditioning regimen but the dose of TBI was reduced to half (1.5 Gy) (FIG. 6). Administration of 10 mg/kg×11 of these Bcl-2 inhibitors, higher Bax expression in T cells, B cells and NK cells was only observed with venetoclax but not with navitoclax (FIG. 7A). As a result, more effective deletion of CD4 and CD8 effector memory T cells (TEM) was observed with venetoclax. In contrast, since navitoclax has affinity to Bcl-xL, which is critical for platelet survival (Kile, B. T. 2014, Br J Haematol 165, 217-226.), prolonged thrombocytopenia was observed after navitoclax treatment (FIG. 7B). These differences in efficacy may be attributed to ABT-199's significantly higher inhibitor constant (Ki) to the target proteins (Ki 0.01 vs. 0.5 nM) (Selleckchem.com). While navitoclax could be more effective if the dose was increased, unacceptable thrombocytopenia would result (FIG. 7B). Because of these efficacy and safety profiles, venetoclax was selected to pursue in subsequent studies.

Results of the Original Regimen without Bcl-2 Inhibition

As summarized in Table 3 below, the original conditioning regimen comprising TBI 3 Gy in addition to anti-CD154 (Group A) or belatacept (Group B), 60-75% survived long-term (>1 year) without ongoing immunosuppression (Table 3), but all recipients required support for 10-12 days of significant pancytopenia. With the reduced TBI dose (1.5 Gy), all four recipients failed to develop chimerism and three of four recipients suffered early T cell mediated rejection (TCMR) (Group C).

TABLE 3
Results of the original regimen and the modified regimen with Bc1-2 inhibition

| | | | TBI | TI | | | | Renal Allograft |
|---|---|---|---|---|---|---|---|---|
| | Group | n | (Gy) | (Gy) | Venetoclax | CoB | Chimerism | Survival (days) |
| No | A | 8 | 3.0 | 7 | − | aCD154 | 7/8 | 2498, 4328, 837, 755, |
| Venetoclax | | | | | | | | 401 373, 206, 58 |
| | B | 5 | 3.0 | 7 | − | Belatacept | 4/5 | 861, 796, 378, 156, 125 |
| | C | 4 | 1.5 | 7 | − | aCD154 | 0/4 | >430, 167, 100, 58 |
| +Venetoclax | D | 5* | 1.5 | 7 | + | aCD154 | 5/5 | >1071, >735, >313 |
| | E | 3 | 1.5 | 7 | + | Belatacept | 3/3 | >574, >308, 237 |
| | F | 2 | 1.5 | 7 | + | — | 0/2 | 74, 127 |
| | G | 3 | 1.5 | 0 | + | aCD154 | 3/3 | 97, 100, 163 |
| | H | 2 | 0 | 7 | + | aCD154 | 0/2 | 120, 142 |

TBI: total body irradiation, TI: thymic irradiation, CoB: costimulatory blockade, aCD154: anti-CD154 mAb,
*bone marrow transplant only in two recipients.

Bcl-2 Inhibition with Venetoclax Induced Effective Deletion of Effector T Cells and B Cells but Preserved Regulatory T Cells

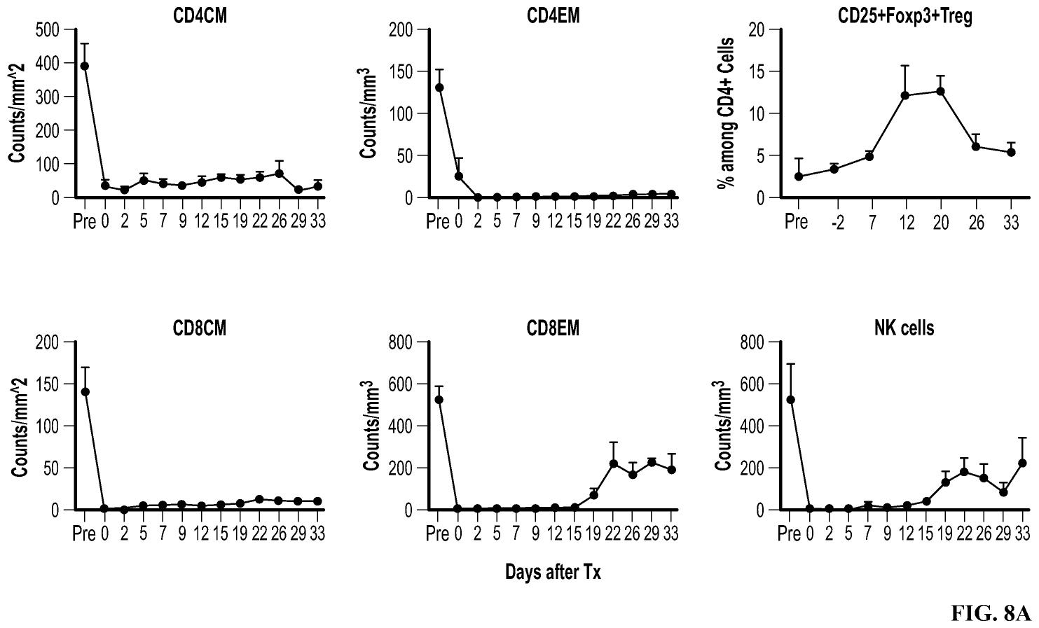

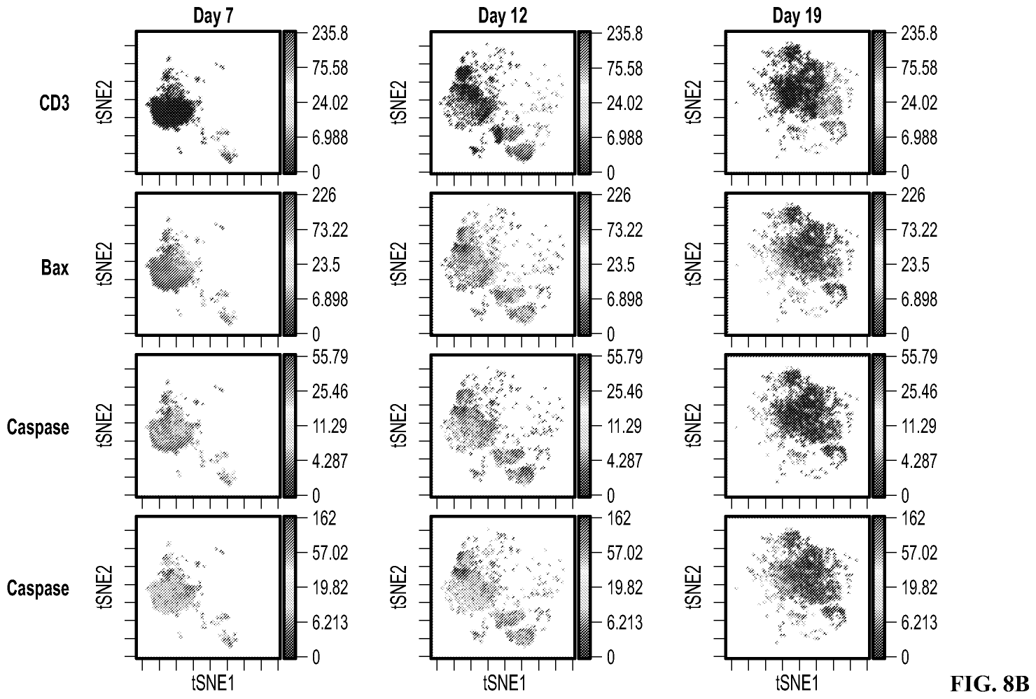

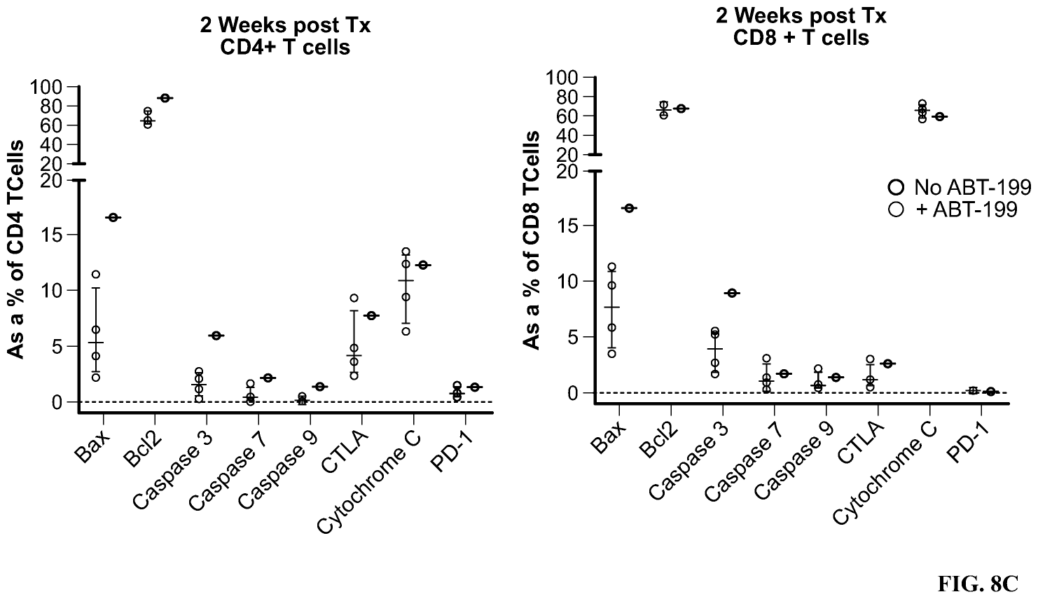

Venetoclax (10 mg/kg×11; administered on days −4 to 6) was added to the reduced TBI conditioning regimen (1.5 Gy) without alterations of other treatments of the original regimen (Groups D or E in Table 3 and FIG. 6). Prompt deletion of CD4⁺ effector memory (EM), CD8⁺ central memory (CM), CD8⁺ EM, and NK cells was observed (FIG. 8A). Some populations of CD4⁺ CM, including regulatory T cells (Tregs), were relatively preserved as expected, since Bcl-2 is a key survival gene for conventional T cells but not for Tregs (Wang, X., et al. 2012, e270.; Issa, F., et al. 2019, Frontiers in immunology 10, 889.). As a result, enrichment of Tregs was observed (FIG. 8A). Expression of various apoptotic markers, such as Bcl-2, Cytochrome C, Bcl-2 associated X (Bax), and Caspases 3, 7 and 9, were analyzed using cytometry time of flight (CyTOF). The t-SNE maps revealed upregulated expression of apoptotic markers until day 12 and then active apoptosis subsided by day 19 (FIG. 8B). During the second week post-transplant, expression of Bax and Caspase 3 was higher in a recipient CD4⁺ and CD8⁺ T cells treated with ABT-199 than in animals not administered venetoclax (FIG. 8C).

Bcl-2 Inhibition with Venetoclax Significantly Promoted Chimerism without Myelosuppression

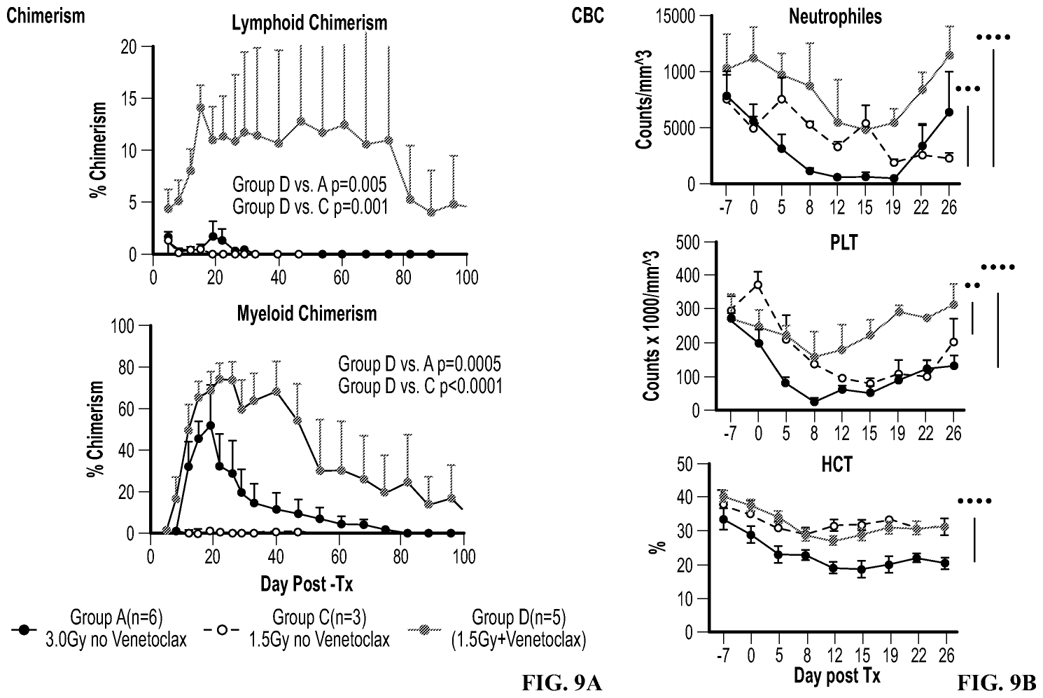

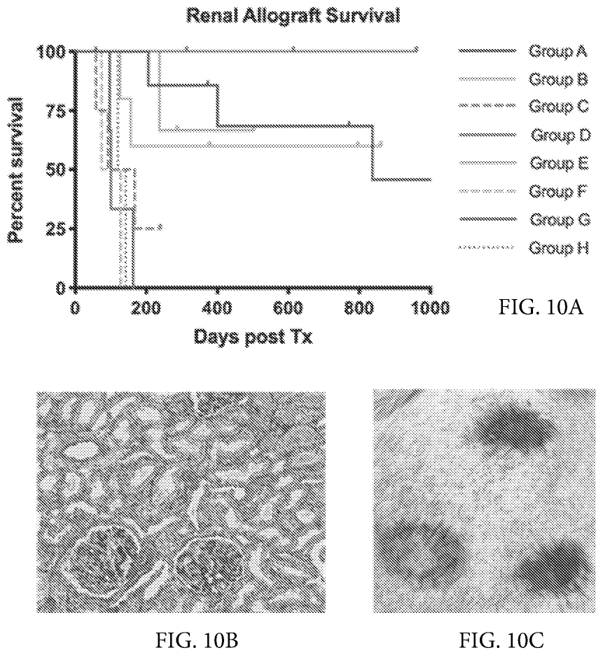

In contrast to recipients not administered venetoclax, all five recipients treated in Group D (TBI 1.5Gy with venetoclax and anti-CD154 mAb) developed chimerism that was significantly higher (p<0.005) and more prolonged over that observed even in Group A recipients administered TBI 3 Gy without venetoclax (FIG. 9A). Of particular relevance to the clinical application, no leukopenia, thrombocytopenia, or anemia were observed in the three Group D recipients administered venetoclax (FIG. 9B). This was in sharp contrast to the pancytopenia observed in Group A recipients treated with TBI 3 Gy without venetoclax (FIG. 9B). Interestingly, the Group C recipients treated with TBI 1.5 Gy but without venetoclax developed delayed leukopenia and thrombocytopenia (FIG. 9B). This may be attributed to failed chimerism induction, in which absence of donor cells hindered the recovery of CBC counts. Kidney transplantation was combined in three recipients in Group D and all three achieved long-term immunosuppression-free allograft survival, with the longest survival exceeding 1000 days (Table 3, FIG. 10A). In association with the high level lymphoid chimerism (Thaiss, C. C., et al. 2019, Transplantation 103, 689-697.), neither chronic rejection nor DSA has been observed in these recipients (FIG. 10B). Skin transplantation performed one year after CKBMT showed specific acceptance of the skin from the kidney and bone marrow donor (FIG. 10C).

Costimulatory Blockade is Essential to Promote Hematopoietic Chimerism with Bcl-2 Inhibition

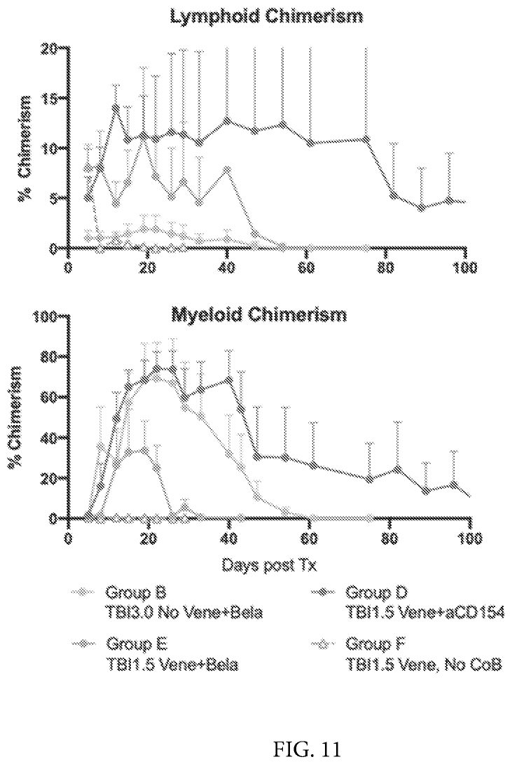

Since anti-CD154 mAb is not currently clinically available, a costimulatory blockade (CoB) with the clinically available CTLA4Ig, belatacept, was tested in place of anti-CD154 (Group E). Although chimerism achieved by Group E recipients was less than that achieved by Group D recipients treated with anti-CD154 mAb, all three Group E recipients treated with belatacept developed multilineage chimerism (FIG. 11). Two of the three Group E recipients are currently surviving long-term (>574 days and >308 days) without any immunosuppression (FIG. 10A). Two recipients without CoB (Group F) failed to develop chimerism and rejected their renal allografts rapidly (Table 3, FIG. 10A), suggesting that CoB is essential to induce chimerism in the approach with Bcl-2 inhibition.

### Further Studies on Radiation Requirement for Induction of Chimerism and Allograft Tolerance

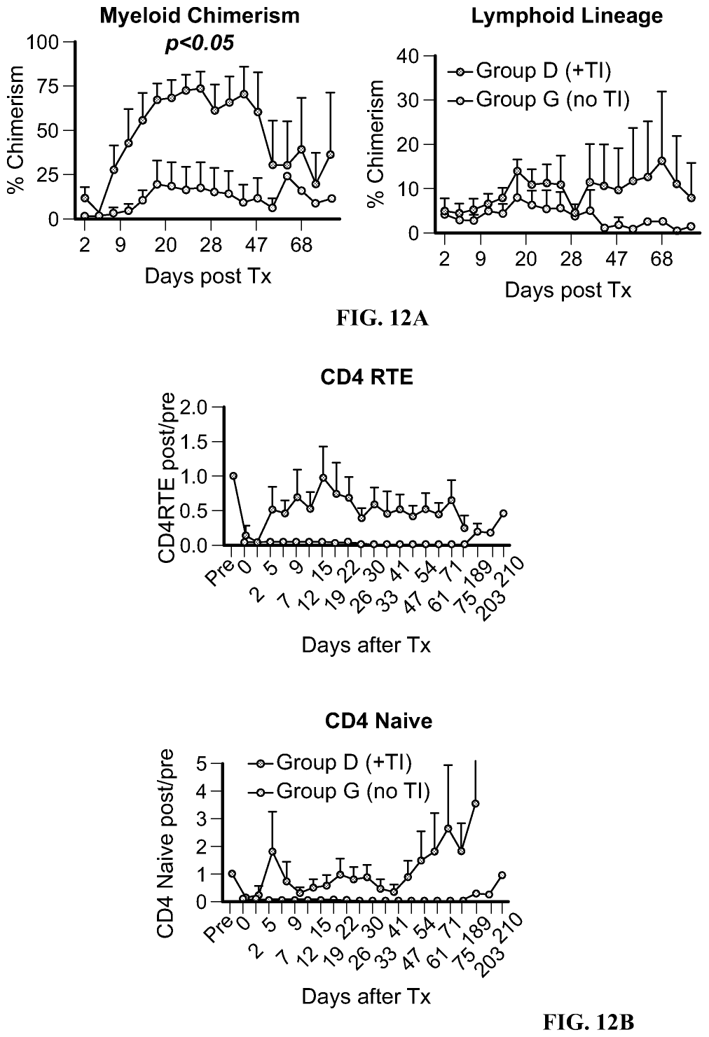

In the murine studies on hematopoietic chimerism by Sharabi et al (Sharabi, Y., et al. 1990, J Exp Med 172, 195-202.), thymic irradiation (TI) was found to be essential to induce stable mixed chimerism and skin allograft tolerance. However, TI was not required in the murine studies with Bcl-2 inhibition by Cippa et al. Three recipients were treated with Group D regimen but without TI (Table 3, Group G). All three recipients developed chimerism but the levels were significantly lower (FIG. 12A) than Group D. However, all Group G recipients rejected their kidney allograft early with donor specific antibodies (DSA). A notable difference between Groups D and G recipients was post-transplant counts of CD4⁺ recent thymic emigrants (RTE) (FIG. 12B) and CD4⁺ naïve T cells, which recovered quickly in the Group G recipients to the pre-transplant levels after two weeks. In contrast, the recovery of RTE and CD4⁺ naïve T cells was not observed until day 200 in Group D recipients.

The treatment regimen without TBI was then examined (Group H). Both recipients of Group H failed to develop chimerism and rejected their allograft early (Table 3, FIG. 10A), suggesting very low dose myelosuppressive therapy is still necessary in the approach with selective Bcl-2 inhibition.

### Example 4: Induction of Hematopoietic Chimerism by MCL-1 and Bcl-2 Inhibition without Chemo/Radiation Therapy in Primates

Despite marked improvement in short-term allograft survival rates over the last three decades, life-long use of immunosuppressive drugs significantly increases the risk of death from cardiovascular disease, infection, and malignancies. In addition, recipients suffer major morbidities, including nephrotoxicity, de novo diabetes, dyslipidemia, and neurotoxicity. Moreover, even with life-long use of potent immunosuppressive drugs, the development of chronic rejection is not consistently prevented. Consequently, the 10-year renal allograft survival rate after deceased donor kidney transplantation (KTx) remains around 50%, with as many as 25% of recipients dying despite ongoing graft function.

Induction of allograft tolerance would be an ideal solution to overcome the limitations of the current therapy. Among the tolerance induction protocols tested to date, induction of transient or durable donor hematopoietic chimerism through DBMT is the only approach that has provided repeatedly successful immunosuppression-free allograft survival for human renal allograft recipients.

Widespread clinical application of current tolerance induction approaches is hampered by the severe myelosuppressive effect of current conditioning therapies (primarily total body irradiation (TBI) or cyclophosphamide) and the resultant pancytopenia, which requires intensive antimicrobial prophylaxis, isolation precautions, and extended hospitalization. Methods have recently been described wherein successful reduction of the requisite doses of total body irradiation (TBI) have been achieved by using Venetoclax (ABT-199), a small molecule inhibitor of the anti-apoptotic protein BCL-2. However, possibly because Bcl-2 inhibition by ABT-199 does not delete host hematopoietic stem cells (HSCs) to empty bone marrow (BM) niches, minimal dose of TBI 1.5 Gy was still required for successful induction of chimerism.

It remains desirable to eliminate the requirement for chemotherapeutic/radiation therapy in the conditioning regimen. Deletion of HSCs to open the physical space in BM niches is considered essential to achieve HSC engraftment. This has been accomplished in various therapeutic regimens by TBI or chemotherapeutic drugs, but has been associated with severe myelosuppression and other side effects. The anti-C-kit (CD117) antibody, which specifically binds to HSCs, has also been reported to effectively delete HSCs and open space in BM niches. However, deletion of HSCs alone does not allow for the achievement of engraftment of allogeneic HSCs; effective suppression of recipient alloimmune responses must also be achieved. To date, there has been no successful induction of hematopoietic chimerism with anti-CD117 mAb in MHC-mismatched BMT in immunocompetent NHP's or humans.

Since HSCs depend on Mcl-1 for their survival, it was hypothesized that inhibition of Mcl-1 will result in selective deletion of HSCs via intrinsic apoptosis. These studies revealed that combination of Mcl-1 and Bcl-2 inhibitors effectively delete HSCs in BM niches and induced multilineage hematopoietic chimerism in MHC mismatched BMT for the first time in nonhuman primates.

Results

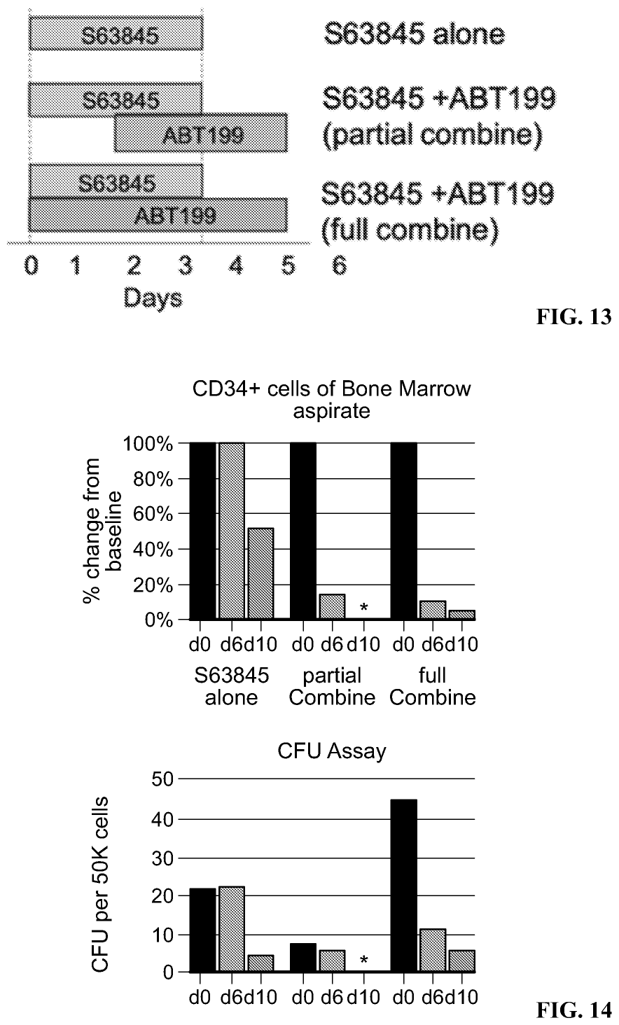

To test HSC-depleting potential by Mcl-1 inhibition, three cynomolgus macaques were treated with either Mcl-1 inhibitor (S63845, 5 mg/kg×5) alone or combined with ABT-199 (FIG. 13). Upon administration of S63845 alone, HSCs were deleted to 51% of the pretreatment level in BM (FIG. 14). A total colony forming units (CFU) were also suppressed to 45% (FIG. 14). Partial combination of ABT-199 (10 mg/kg×11 doses, starting 3 days after S63845) with S63845 resulted in the decrease of HSCs to 22% by day 6 (FIG. 14). Full combination of ABT-199 with S63845 resulted in significant deletion of HSCs (2.2%) with effectively decreased CFU (11%) (FIG. 14).

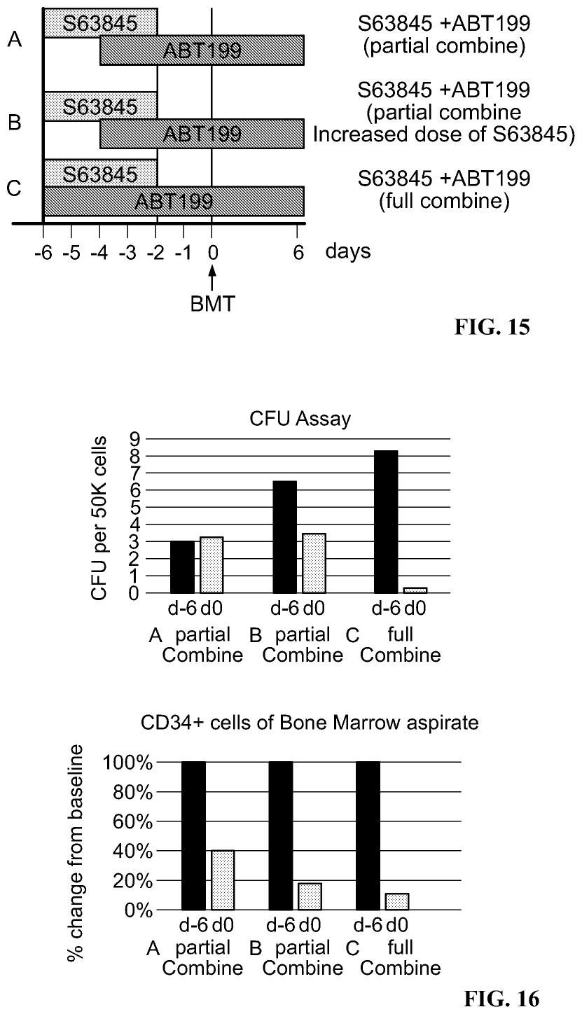

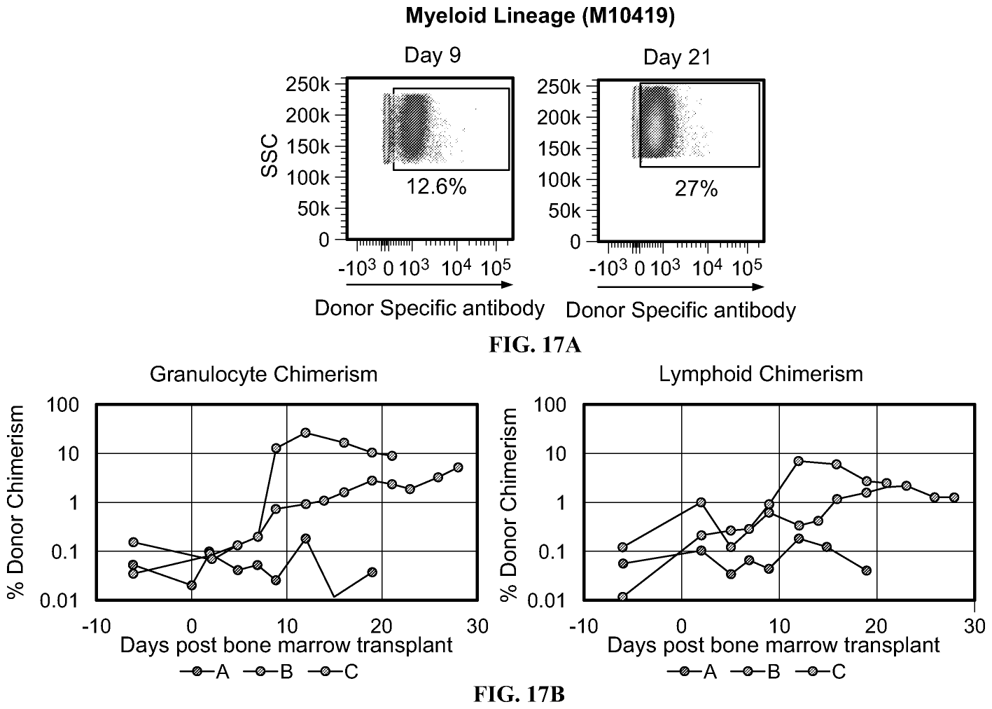

Based on these drug-only studies, BMT from a MHC-mismatched donor was performed after conditioning with S63945 and ABT-199 in three monkeys. To maximize the chance of successful induction of chimerism, all recipients were also treated with pre-BMT ATG and post BMT treatments with anti-CD154 (up to day 14 post BMT) and cyclosporine (up to day 28), all of which have been previously show to be required for chimerism induction. Partial combination of ABT-199 with a Mcl-1 inhibitor (Mcl-1i), Regimen A (FIG. 15), resulted in deletion of HSCs to 40% (FIG. 16) and no hematopoietic chimerism was induced. The dose of Mcl-1i was increased to 7.5 mg/kg administered on days −3 and −2 pre-BMT (Regimen B). Regimen B resulted in suppressed CFUs and HSCs were deleted to 16% (FIG. 16). The monkey administered Regimen B started to have small levels of multilineage chimerism on day 9, after which it slowly increased to 9% by day 28 (FIG. 16). Lymphoid chimerism is also detectable as high as 2.1% (FIG. 17). Upon simultaneous administration of S63845 and ABT-199 (FIG. 15, Regimen C), almost complete deletion of HSCs was achieved in BM with marked suppression of CFU (3%). The recipient of Regimen C started to develop multilineage chimerism on day 7, after which it increased to as high as 27% in the myeloid lineage (FIG. 16). Lymphoid chimerism also reached to 6% by day 24. Recipients of Regimen B and Regimen C remain chimeric as long as 110 days (still in progress). Neither S63845 or the S63845/ABT-199 combination showed significant toxicity but mild transient transaminitis and elevated amylase was observed.

### CONCLUSION

Combined inhibition of Mcl-1 and Bcl-2 resulted in significant HSC deletion, which made hematopoietic chimerism induction possible without any chemotherapeutic/radiation therapy.

1. A method for inducing hematopoietic chimerism in a patient in need thereof, wherein the method comprises:
a. administering an inhibitor of an anti-apoptotic Bcl-2 family member to the patient;
b. administering total body irradiation (“TBI”) to the patient; and
c. transplanting bone marrow from a donor to the patient;
such that hematopoietic chimerism is induced in the patient.

2. A method for inducing hematopoietic chimerism in a patient in need thereof, wherein the method comprises:
a. administering a specific inhibitor of Bcl-2; and
b. transplanting bone marrow from a donor to the patient;
such that hematopoietic chimerism is induced in the patient.

3. A method for inducing hematopoietic chimerism in a patient in need thereof, wherein the method comprises:
a. administering a first course of a first inhibitor of an anti-apoptotic Bcl-2 family member to the patient;
b. transplanting bone marrow from a donor to the patient; and
c. administering a second course of a second inhibitor of an anti-apoptotic Bcl-2 family member to the patient starting 1, 2, 3, 4, 5, 6, 7, 8, 9, or 10 days after completion of the first course, such that hematopoietic chimerism is induced in the patient.

4. The method of claim 3, wherein administering the second course of the second inhibitor of an anti-apoptotic Bcl-2 family member to the patient starts between 2 days to 5 days after completion of the first course.

5. A method for inducing hematopoietic chimerism in a patient in need thereof, wherein the method comprises administering an inhibitor of an anti-apoptotic Bcl-2 family member to the patient, wherein the patient is not being treated with an anti-CD154 antibody.

6. A method for inducing hematopoietic chimerism in a patient in need thereof, wherein the method comprises administering an inhibitor of an anti-apoptotic Bcl-2 family member to the patient, wherein the inhibitor is not ABT-737.

7. A method for inducing hematopoietic chimerism in a patient in need thereof, wherein the method comprises:
a. administering an inhibitor of an anti-apoptotic Bcl-2 family member to the patient;
b. administering total body irradiation (“TBI”) to the patient; and
c. transplanting bone marrow and an organ selected from heart, intestine, kidney, or liver from a donor to the patient.

8. The method of claim 3, wherein the first and the second inhibitor are the same.

9. The method of claim 3, wherein the treatment regimen reduces the risk of Graft versus Host Disease (GVHD) in the recipient.

10. The method of claim 3, or 9 wherein the treatment regimen reduces the incidence of GVHD by about 5%, 10%, 15%, 20%, 25%, 30%, 35%, 40%, 45%, 50%, 55%, 60%, 65%, 70%, 75%, or 80%, 85%, 95% as compared to no treatment regimen in the recipient.

11. The method of claim 3, or 9, wherein the treatment regimen reduces the severity and/or degree of GVHD in the recipient by about 5%, 10%, 15%, 20%, 25%, 30%, 35%, 40%, 45%, 50%, 55%, 60%, 65%, 70%, 75%, or 80%, 85%, 95% as compared to no treatment regimen in the recipient.

12. The method of claim 1, wherein the dose of TBI is 0.5 Gy, 0.75 Gy, 1.0 Gy, 1.25 Gy, 1.50 Gy, 1.75 Gy, 2.0 Gy, 2.25 Gy, 2.50 Gy, or 3 Gy.

13. The method of claim 1, wherein the dose of TBI is 1.5 Gy

14. The method of claim 1 or claim 3, wherein the anti-apoptotic Bcl-2 family member is Bcl-2.

15. The method of claim 2, wherein the specific inhibitor of Bcl-2 is venetoclax, oblimersen, PNT2258, or SPC2996.

16. The method of claim 15, wherein the specific inhibitor of Bcl-2 is venetoclax.

17. The method of claim 1 or claim 3, wherein the inhibitor of an anti-apoptotic BCL-2 family member is a combination of inhibitors of Mcl-1 and Bcl-2.

18. The method of claim 17, wherein the inhibitor of Mcl-1 is obatoclax, A-1210477, AMG176, S64315, S63845, or AZD5991.

19. The method of claim 17, wherein the inhibitor of Mcl-1 is S63845 or S64315.

20. The method of claim 10, wherein chimerism can be induced without need of myelosuppressive treatments (e.g., cyclophosphamide or TBI).

21. The method of any one of claim 1 to claim 3, wherein the method does not result in neutropenia.

22. The method of any one of claim 1 to claim 3, wherein the method results in a nadir (lowest levels) of neutrophil counts no less than 100/mm³, 200/mm³, 300/mm³, 400/mm³, 500/mm³, 600/mm³, 700/mm³, 800/mm³, 900/mm³, 1000/mm³, 1500/mm³, 1600/mm³, 1700/mm³, 1800/mm³, 1900/mm³, 2000/mm³, before the method.

23. The method of any one of claim 1 to claim 3, wherein the method results in a nadir (lowest levels) of neutrophil counts no less than 1500/mm³, while they are decreased to less than 100/mm³ with the previous methods.

24. The method of any one of claim 1 to claim 3, wherein the hematopoietic chimerism is characterized by a percentage of donor cells in the lymphohematopoietic system of at least 5%, 10%, 15%, 20%, 25%, 30%, 35%, 40%, 45%, 50%, 55%, 60%, 65%, 70%, 75%, 80%, 85%, or at least 90%.

25. The method of any one of claim 1 to claim 3, wherein the hematopoietic chimerism is characterized by a percentage of donor cells in the lymphohematopoietic system of at least 10%.

26. The method of claim 1 or claim 2, wherein the hematopoietic chimerism is transient or persists indefinitely.

27. The method of any one of claim 1 to claim 3, wherein the method is performed such that immune reconstitution is essentially complete, or within the normal reference range, at most 2 months, 3 months, 4 months, 5 months, 6 months, 7 months, 8 months, 9 months, 10 months, 11 months, or at most 1 year after completion of the method.

28. The method of any one of claim 1 to claim 3, wherein the method is performed such that immune reconstitution is essentially complete, or within the normal reference range, at most 2 months.

29. The method of any one of claim 1 to claim 3, wherein the inhibitor is administered to the patient for 10 doses of 10 mg/kg, 11 doses of 10 mg/kg, 12 doses of 10 mg/kg, 13 doses of 10 mg/kg, 14 doses of 10 mg/kg, or 15 doses of 10 mg/kg.

30. The method of any one of claim 1 to claim 3, wherein the inhibitor is administered to the patient for 11 doses of 10 mg/kg.

31. The method of any one of claim 1 to claim 3, wherein the inhibitor is administered to the patient at a daily dose of 10 mg/kg.

32. The method of any one of claim 1 to claim 3, wherein the inhibitor is administered to the patient intravenously, intravascularly, topically, intraarterially, intracranially, intramuscularly, orally, intraorbitally, by inhalation, transdermally, or intraperitonially.

33. The method of any one of claim 1 to claim 3, wherein the inhibitor is administered to the patient orally.

34. The method of any one of claim 1 to claim 3, wherein the method further comprises administering 1 Gy, 2 Gy, 3 Gy, 4 Gy, 5 Gy, 6 Gy, 7 Gy, 8 Gy, 9 Gy, 10 Gy, 11 Gy, 12 Gy, 13 Gy, 14 Gy, 15 Gy, 16 Gy, 12 Gy, 18 Gy, 19 Gy, or 20 Gy of local thymic irradiation to the patient for induction of allograft tolerance.

35. The method of any one of claim 1 to claim 3, wherein the method further comprises administering 7 Gy of local thymic irradiation to the patient for induction of allograft tolerance.

36. The method of any one of claim 1 to claim 3, wherein the method further comprises administering cyclosporine A to the patient.

37. The method of any one of claim 1 to claim 3, wherein the method further comprises administering Anti-thymocyte globulin (“ATG”) to the patient.

38. The method of claim 36, wherein the ATG is administered to the patient for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days.

39. The method of claim 36, wherein the ATG is administered to the patient at a daily dose of about 0.5 mg/kg, about 1 mg/kg, about 1.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg.

40. The method of claim 23, wherein the ATG is administered to the patient intravenously, intravascularly, intraarterially, intracranially, intramuscularly, intraorbitally, transdermally, or intraperitonially.

41. The method of any one of claims 1 to 40, wherein the method further comprises transplanting an organ from an organ-donor to the patient.

42. The method of claim 41, wherein the organ-donor and the donor of the bone marrow are the same person or the donor of the bone marrow is a person that is HLA-identical to the organ-donor.

43. The method of claim 41, wherein the organ is a heart, intestine, kidney, liver, lung, or a pancreas.

44. The method of claim 41, wherein the organ is HLA mismatched.

45. A method for performing a bone marrow transplant, wherein the method comprises administering to the recipient of the bone marrow transplant an inhibitor of Mcl-1.

46. The method of claim 45 wherein the inhibitor of Mcl-1 is a specific inhibitor of Mcl-1.

47. The method of claim 45 wherein the inhibitor of Mcl-1 is administered with an inhibitor of a specific inhibitor of Bcl-2.

48. The method of claim 45 wherein the inhibitor of Mcl-1 is obatoclax, A-1210477, AMG176, S64315, S63845, or AZD5991.

49. The method of claim 45 wherein the inhibitor of Mcl-1 is administered at about 10 mg, about 20 mg, about 30 mg, about 40 mg, about 50 mg, about 60 mg, about 70 mg, about 80 mg, about 90 mg, about 100 mg, about 150 mg, about 200 mg, about 300 mg, about 400 mg, about 500 mg, about 600 mg, about 700 mg, about 800 mg, about 900 mg, or about 1000 mg.

50. The method of any one of claims 1 to 3, wherein the method further comprises administering an Mcl-1 inhibitor at a dose sufficient to reduce recipient's hematopoietic stem cell in niches.

51. The method of claim 1 or 2, wherein the inhibitor of an anti-apoptotic BCL-2 family member is an inhibitor of Mcl-1.

52. The method of claim 51, wherein the inhibitor of Mcl-1 is administered as a monotherapy.

53. The method of any one of claim 3, wherein the initial administration of the first anti-apoptotic BCL-2 family member to the patient occurs at the same time as the initial administration of the second anti-apoptotic BCL-2 family member to the patient.

54. The method of claim 53, wherein the first anti-apoptotic BCL-2 family member is venetoclax (ABT-199) and the second anti-apoptotic BCL-2 family member is S63845.

55. A method for inducing hematopoietic chimerism in a patient in need thereof, wherein the method comprises:
a. administering an inhibitor of Mcl-1 to the patient;
b. administering total body irradiation (“TBI”) to the patient; and
c. transplanting bone marrow from a donor to the patient;
such that hematopoietic chimerism is induced in the patient.

56. A method for inducing hematopoietic chimerism in a patient in need thereof, wherein the method comprises:
a. administering an inhibitor of Mcl-1 to the patient; and
b. transplanting bone marrow from a donor to the patient;
such that hematopoietic chimerism is induced in the patient.

57. The method of claim 55 or 56, wherein the inhibitor of Mcl-1 is obatoclax, A-1210477, AMG176, S64315, S63845, or AZD5991.

58. The method of claim 57 wherein the inhibitor of Mcl-1 is S63845.

59. The method of any one of claims 1 to 58, wherein the chimerism can last at least 1 month, 2 months, 3 months, 4 months, 5 months, or at least 6 months.

60. The method of any one of claims 1 to 58, wherein the chimerism can last at least 6 months.
